(19) United States
(12) Patent Application Publication
(10) Pub. No.: US 2023/0312756 A1
(43) Pub. Date: Oct. 5, 2023

(54) METHODS FOR THE USE OF A PD-1 X CTLA-4 BISPECIFIC MOLECULE

(71) Applicant: MACROGENICS, INC., Rockville, MD (US)

(72) Inventors: Bradley James SUMROW, Rockville, MD (US); Ezio BONVINI, Rockville, MD (US); Sharad SHARMA, Rockville, MD (US); Jon Marc WIGGINTON, Rockville, MD (US); Alexey Yevgenyevich BEREZHNOY, Rockville, MD (US)

(21) Appl. No.: 18/006,294

(22) PCT Filed: Jul. 23, 2021
(86) PCT No.: PCT/US2021/042901

§ 371 Date: Jan. 20, 2023

Related U.S. Application Data
(60) Provisional application No. 63/057,054, filed on Jul. 27, 2020.
(60) Provisional application No. 63/177,036, filed on Apr. 20, 2021.
(60) Provisional application No. 63/219,066, filed on Jul. 7, 2021.

(51) Int. Cl. C07K 16/46; A61P 35/00; A61P 37/04

(52) U.S. Cl. CPC C07K 16/468; A61P 35/00; A61P 37/04; C07K 2317/31; C07K 2317/565; C07K 2317/53; C07K 2317/626

(57) ABSTRACT

The present invention is directed in part to dosing regimens for administering a PD-1×CTLA-4 bispecific molecule for the treatment of cancer, and other conditions. The invention is directed in part to the use of such molecules, and to the use of pharmaceutical compositions and pharmaceutical kits that contain such molecules and that facilitate the use of such dosing regimens in the treatment of cancer or to stimulate immune cells.

### CROSS-REFERENCE TO RELATED APPLICATIONS

This application claims priority to U.S. patent application No. 63/057,054 (filed on Jul. 27, 2020; U.S. patent application No. 63/177,036 (filed on Apr. 20, 2021), and U.S. patent application No. 63/219,066 (filed on Jul. 7, 2021), each of which is incorporated herein by reference in its entirety for all purposes.

### REFERENCE TO SEQUENCE LISTING

This application includes a Sequence Listing pursuant to 37 C.F.R. 1.821 et seq., which has been submitted electronically in ASCII format and is hereby incorporated by reference in its entirety for all purposes. The ASCII copy of the Sequence Listing, created on Jul. 15, 2021, is named MAC-0115-PC_SL.txt and is 30,796 bytes in size.

### FIELD OF THE INVENTION

The present invention is directed in part to dosing regimens for administering a PD-1×CTLA-4 bispecific molecule for the treatment of cancer and other diseases and conditions. The present invention also pertains in part to methods of using such PD-1×CTLA-4 bispecific molecules to stimulate immune cells. The invention in part concerns the use of such regimens for the administration of tetravalent PD-1×CTLA-4 bispecific diabodies that comprise two binding sites for PD-1 and two binding sites for CTLA-4. The invention is directed in part to the use of such bispecific molecules. The invention is also directed in part to the use of pharmaceutical compositions and pharmaceutical kits that contain such molecules, which facilitate the use of such dosing regimens in the treatment of cancer or to stimulate immune cells.

### BACKGROUND OF THE INVENTION

### I. CTLA-4

Cytotoxic T-lymphocyte associated protein-4 (CTLA-4; CD152) is a single pass type I membrane protein that forms a disulfide linked homo-dimer dimer (Schwartz J. C., et al. (2001) “Structural Basis For Co-Stimulation By The Human CTLA-4 B7-2 Complex,” Nature 410:604-608). CTLA-4 is primarily an intracellular antigen whose surface expression is tightly regulated by restricted trafficking to the cell surface and rapid internalization. CTLA-4 acts as a negative regulator of T effector cell activation that diminishes effector function and dictates the efficacy and duration of a T-cell response (Linsley, P. S. et al. (1996) “Intracellular Trafficking Of CTLA-4 And Focal Localization Towards Sites Of TCR Engagement,” Immunity 4:535-543). Blockage of CTLA-4 is reported to enhance T-cell responses in vitro and also to increase antitumor immunity. Thus, blockage of CTLA-4 using anti-CTLA-4 antibodies has been proposed to provide new treatments for disease, especially human diseases where immune stimulation might be beneficial such as for treatment of cancers and infectious diseases (see, Leach, D. R., et al. (1996) “Enhancement Of Antitumor Immunity By CTLA-4 Blockade,” Science. 271:1734-1736; WO 01/14424; WO 00/37504). Development of blockers of CTLA-4 function has focused on the use of monoclonal antibodies such as ipilimumab (see, e.g., Hodi, F. S., et al., (2003) “Biologic Activity Of Cytotoxic T Lymphocyte-Associated Antigen 4 Antibody Blockade In Previously Vaccinated Metastatic Melanoma And Ovarian Carcinoma Patients,” Proc. Natl. Acad. Sci. (U.S.A.) 100:4717-4717) and tremelimumab (Ribas, A. et al. (2005) “Antitumor Activity In Melanoma And Anti-Self Responses In A Phase I Trial With The Anti-Cytotoxic T Lymphocyte-Associated Antigen 4 Monoclonal Antibody CP-675,206,” Oncologist 12: 873-883).

### II. PD-1

Programmed Death-1 (“PD-1,” also known as “CD279”) is an approximately 31 kD type I membrane protein member of the extended CD28/CTLA-4 family of T-cell regulators that broadly negatively regulates immune responses (Ishida, Y. et al. (1992) “Induced Expression Of PD-1, A Novel Member Of The Immunoglobulin Gene Superfamily, Upon Programmed Cell Death,” EMBO J. 11:3887-3895. PD-1 mediates its inhibition of the immune system by binding to the transmembrane protein ligands: Programmed Death-Ligand 1 (“PD-L1,” also known as “B7-H1”) and Programmed Death-Ligand 2 (“PD-L2,” also known as “B7-DC”) (Flies, D. B. et al. (2007) “The New B7s: Playing a Pivotal Role in Tumor Immunity,” J. Immunother. 30(3):251-260).

The role of PD-1 ligand interactions in inhibiting activation and/or proliferation of T cells has suggested that these biomolecules might serve as therapeutic targets for treatments of inflammation and cancer. The use of anti-PD-1 antibodies to treat tumors and up-modulate an adaptive immune response has been proposed and antibodies capable of specifically binding to PD-1 have been reported (see, e.g., Patnaik A. et al. (2015) “Phase I Study of Pembrolizumab (MK-3475; Anti-PD-1 Monoclonal Antibody) in Patients with Advanced Solid Tumors,” Clin Cancer Res; 21(19):4286-4293; U.S. Pat. Nos. 7,488,802; 7,521,051; 7,595,048; 8,008,449; 8,354,509; 8,735,553; 8,779,105; 8,900,587; 9,084,776; 9,815,897; 10,577,422; WO 2014/194302; and WO 2015/035606; WO 2004/056875; WO 2006/121168; WO 2008/156712; WO 2012/135408; WO 2012/145493; WO 2013/014668; WO 2014/179664; WO 2014/194302; WO 2015/112800; and WO 2019/246110).

Combination therapy using separate intravenous doses of the anti-CTLA-4 antibody ipilimumab and the anti-PD-1 antibody nivolumab with chemotherapy have recently been approved for the treatment of for certain patients with metastatic or recurrent non-small cell lung cancer (NSCLC). However, combination therapy was accompanied by increased frequency and severity of treatment-related adverse events (TRAEs). Fifty-five percent of patients receiving the combination of ipilimumab and nivolumab experienced severe TRAEs, a significant increase compared to 16% for nivolumab alone and 27% for ipilimumab alone (Larkin, J., et al., 2015. “Combined Nivolumab and Ipilimumab or Monotherapy in Untreated Melanoma,” N. Engl. J. Med.). Beyond the potential medical consequences of severe TRAEs for cancer patients, TRAEs often necessitate discontinuation of treatment, limiting the therapeutic benefit in these populations.

Bispecific molecules binding to both PD-1 and CTLA-4 allow for great flexibility in the design and engineering in various applications, providing enhanced avidity to multimeric antigens, the cross-linking of differing antigens, and directed targeting to specific cell types relying on the presence of both target antigens. The use of PD-1×CTLA-4 bispecific molecules in the treatment of cancer has been proposed and PD-1×CTLA-4 bispecific molecules have been described for example in WO 2014/209804; WO 2017/218707; WO 2017/193032; WO 2019/094637; and US 2019/0185569. In particular, tetravalent PD-1×CTLA-4 bispecific diabodies and trivalent PD-1×CTLA-4 binding molecules having exemplary activity are described in WO 2017/106061.

### SUMMARY OF THE INVENTION

Provided are dosing regimens for the administration of PD-1×CTLA-4 bispecific molecules for the treatment of cancer and other diseases and conditions that can minimize undesirable side effects. The present invention also pertains in part to methods of using such PD-1×CTLA-4 bispecific molecules to stimulate immune cells. The invention concerns in part the use of such regimens for the administration of tetravalent PD-1×CTLA-4 bispecific diabodies that comprise two binding sites for PD-1 and two binding sites for CTLA-4. The invention is directed in part to the use of such bispecific molecules. The invention is also directed in part to the use of pharmaceutical compositions and pharmaceutical kits that contain such molecules, which facilitate the use of such dosing regimens in the treatment of cancer or to stimulate immune cells.

In detail, the invention provides a method of treating cancer comprising administering a PD-1×CTLA-4 bispecific molecule to a subject in need thereof, wherein the PD-1×CTLA-4 bispecific molecule comprises at least one PD-1 Binding Domain and at least one CTLA-4 Binding Domain, and wherein the method comprises administering the PD-1×CTLA-4 bispecific molecule to the subject at a dose of from about 3 mg/kg to about 10 mg/kg once every 3 weeks. The invention further provides an embodiment of such method, wherein the PD-1×CTLA-4 bispecific molecule is administered to the subject at a dose of from about 3 mg/kg to about 10 mg/kg once every 3 weeks during an induction period.

The invention further provides a method of stimulating immune cells comprising administering a PD-1×CTLA-4 bispecific molecule to a subject in need thereof, wherein the PD-1×CTLA-4 bispecific molecule comprises at least one PD-1 Binding Domain and at least one CTLA-4 Binding Domain, and wherein the method comprises administering the PD-1×CTLA-4 bispecific molecule to the subject at a dose of from about 3 mg/kg to about 10 mg/kg once every 3 weeks. The invention further provides an embodiment of such method, wherein the PD-1×CTLA-4 bispecific molecule is administered to the subject at a dose of from about 3 mg/kg to about 10 mg/kg once every 3 weeks during an induction period. The invention particularly provides the embodiment of such methods, wherein the immune cells are T cells.

The invention further provides the embodiment of such methods, wherein:
(I) the PD-1 Binding Domain comprises a Light Chain Variable Domain (VL_{PD-1}) that comprises the CDRL1, CDRL2 and CDRL3 of SEQ ID NO:1, and a Heavy Chain Variable Domain (VH_{PD-1}) that comprises the PD-1-specific CDRH1, CDRH2 and CDRH3 of SEQ ID NO:5; and
(II) the CTLA-4 Binding Domain comprises a Light Chain Variable Domain (VL_{CTLA-4}) that comprises the CDRL1, CDRL2 and CDRL3 of SEQ ID NO:9, and a Heavy Chain Variable Domain (VH_{CTLA-4}) that comprises the CTLA-4-specific CDRH1, CDRH2 and CDRH3 of SEQ ID NO:13.

The invention further provides the embodiment of such methods, wherein the PD-1×CTLA-4 bispecific molecule comprises:
(I) two of the PD-1 binding domains; and
(II) two of the CTLA-4 binding domains.

The invention further provides the embodiment of such methods, wherein the PD-1 binding domain comprises the VL Domain of SEQ ID NO:1 and the VH Domain of SEQ ID NO:5.

The invention further provides the embodiment of such methods, wherein the CTLA-4 binding domain comprises the VL Domain of SEQ ID NO:9 and the VH Domain of SEQ ID NO:13.

The invention further provides the embodiment of such methods, wherein the PD-1×CTLA-4 bispecific molecule comprises an Fc Region. The invention particularly provides the embodiment of such methods, wherein the Fc Region is of the IgG1, IgG2, IgG3, or IgG4 isotype.

The invention further provides the embodiment of such methods, wherein the PD-1×CTLA-4 bispecific molecule further comprises a Hinge Domain.

The invention further provides the embodiment of such methods, wherein the Fc Region and the Hinge Domain are both of the IgG4 isotype, and wherein the Hinge Domain comprises a stabilizing mutation.

The invention further provides the embodiment of such methods, wherein the Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

The invention further provides the embodiment of such methods, wherein the modifications that reduces the affinity of the variant Fc Region for an FcγR comprise the substitution of L234A; L235A; or L234A and L235A, wherein the numbering is that of the EU index as in Kabat.

The invention further provides the embodiment of such methods, wherein the modifications that enhances the serum half-life of the variant Fc Region comprise the substitution of M252Y; M252Y and S254T; M252Y and T256E; M252Y, S254T and T256E; or K288D and H435K, wherein the numbering is that of the EU index as in Kabat.

The invention further provides the embodiment of such methods, wherein the PD-1×CTLA-4 bispecific molecule is a diabody comprising one polypeptide chain that comprises the amino acid sequence of SEQ ID NO:40 and a second polypeptide chain that comprises the amino acid sequence of SEQ ID NO:41.

The invention further provides the embodiment of such methods, wherein the PD-1×CTLA-4 bispecific molecule is a diabody comprising two polypeptide chains each comprising the amino acid sequence of SEQ ID NO:40 and two polypeptide chains each comprising the amino acid sequence of SEQ ID NO:41.

Also provided is an embodiment of such methods, in which the PD-1×CTLA-4 bispecific molecule is administered at a dose of between about 3 mg/kg and 8 mg/kg.

The invention further provides the embodiment of such methods, wherein the PD-1×CTLA-4 bispecific molecule is administered at a dose of between about 6 mg/kg and about 10 mg/kg.

The invention further provides the embodiment of such methods, wherein the PD-1×CTLA-4 bispecific molecule is administered at a dose of about 6 mg/kg.

The invention further provides the embodiment of such methods, wherein the PD-1×CTLA-4 bispecific molecule is administered at a dose of about 7 mg/kg.

The invention further provides the embodiment of such methods, wherein the PD-1×CTLA-4 bispecific molecule is administered at a dose of about 8 mg/kg.

The invention further provides the embodiment of such methods, wherein the PD-1×CTLA-4 bispecific molecule is administered at a dose of about 9 mg/kg.

The invention further provides the embodiment of such methods, further comprising administering the PD-1×CTLA-4 bispecific molecule to the subject at a dose of from about 3 mg/kg to about 10 mg/kg once every 6 weeks during a maintenance period, wherein the maintenance period follows the induction period.

The invention further provides the embodiment of such methods, wherein the induction period has a duration of up to about 24 weeks.

The invention further provides the embodiment of such methods, wherein the maintenance period has a duration of at least 6 weeks. The invention particularly provides the embodiment of such methods, wherein the maintenance period has a duration of at least 84 weeks.

The invention further provides the embodiment of such methods, wherein the PD-1×CTLA-4 bispecific molecule is administered at a dose of between about 3 mg/kg and about 8 mg/kg during the induction period.

The invention further provides the embodiment of such methods, wherein the PD-1×CTLA-4 bispecific molecule is administered at a dose of between about 6 mg/kg and about 10 mg/kg during the induction period.

The invention further provides the embodiment of such methods, wherein the PD-1×CTLA-4 bispecific molecule is administered at a dose of about 3 mg/kg during the induction period.

The invention further provides the embodiment of such methods, wherein the PD-1×CTLA-4 bispecific molecule is administered at a dose of about 4 mg/kg during the induction period.

The invention further provides the embodiment of such methods, wherein the PD-1×CTLA-4 bispecific molecule is administered at a dose of about 5 mg/kg during the induction period.

The invention further provides the embodiment of such methods, wherein the PD-1×CTLA-4 bispecific molecule is administered at a dose of about 6 mg/kg during the induction period.

The invention further provides the embodiment of such methods, wherein the PD-1×CTLA-4 bispecific molecule is administered at a dose of about 6.5 mg/kg during the induction period.

The invention further provides the embodiment of such methods, wherein the PD-1×CTLA-4 bispecific molecule is administered at a dose of about 7 mg/kg during the induction period.

The invention further provides the embodiment of such methods, wherein the PD-1×CTLA-4 bispecific molecule is administered at a dose of about 7.5 mg/kg during the induction period.

The invention further provides the embodiment of such methods, wherein the PD-1×CTLA-4 bispecific molecule is administered at a dose of about 8 mg/kg during the induction period.

The invention further provides the embodiment of such methods, wherein the PD-1×CTLA-4 bispecific molecule is administered at a dose of about 8.5 mg/kg during the induction period.

The invention further provides the embodiment of such methods, wherein the PD-1×CTLA-4 bispecific molecule is administered at a dose of about 9 mg/kg during the induction period.

The invention further provides the embodiment of such methods, wherein the PD-1×CTLA-4 bispecific molecule is administered at a dose of about 9.5 mg/kg during the induction period.

The invention further provides the embodiment of such methods, wherein the PD-1×CTLA-4 bispecific molecule is administered at a dose of about 10 mg/kg during the induction period.

The invention further provides the embodiment of such methods, wherein the PD-1×CTLA-4 bispecific molecule is administered at a dose of between about 3 mg/kg and 8 mg/kg during the maintenance period.

The invention further provides the embodiment of such methods, wherein the PD-1×CTLA-4 bispecific molecule is administered at a dose of between about 6 mg/kg and about 10 mg/kg during the maintenance period.

The invention further provides the embodiment of such methods, wherein the PD-1×CTLA-4 bispecific molecule is administered at a dose of about 3 mg/kg during the maintenance period.

The invention further provides the embodiment of such methods, wherein the PD-1×CTLA-4 bispecific molecule is administered at a dose of about 4 mg/kg during the maintenance period.

The invention further provides the embodiment of such methods, wherein the PD-1×CTLA-4 bispecific molecule is administered at a dose of about 5 mg/kg during the maintenance period.

The invention further provides the embodiment of such methods, wherein the PD-1×CTLA-4 bispecific molecule is administered at a dose of about 6 mg/kg during the maintenance period.

The invention further provides the embodiment of such methods, wherein the PD-1×CTLA-4 bispecific molecule is administered at a dose of about 6.5 mg/kg during the maintenance period.

The invention further provides the embodiment of such methods, wherein the PD-1×CTLA-4 bispecific molecule is administered at a dose of about 7 mg/kg during the maintenance period.

The invention further provides the embodiment of such methods, wherein the PD-1×CTLA-4 bispecific molecule is administered at a dose of about 7.5 mg/kg during the maintenance period.

The invention further provides the embodiment of such methods, wherein the PD-1×CTLA-4 bispecific molecule is administered at a dose of about 8 mg/kg during the maintenance period.

The invention further provides the embodiment of such methods, wherein the PD-1×CTLA-4 bispecific molecule is administered at a dose of about 8.5 mg/kg during the maintenance period.

The invention further provides the embodiment of such methods, wherein the PD-1×CTLA-4 bispecific molecule is administered at a dose of about 9 mg/kg during the maintenance period.

The invention further provides the embodiment of such methods, wherein the PD-1×CTLA-4 bispecific molecule is administered at a dose of about 9.5 mg/kg during the maintenance period.

The invention further provides the embodiment of such methods, wherein the PD-1×CTLA-4 bispecific molecule is administered at a dose of about 10 mg/kg during the maintenance period.

The invention further provides the embodiment of such methods, wherein the dose of PD-1×CTLA-4 bispecific molecule administered in the maintenance period is the same as the dose administered in the induction period.

The invention further provides the embodiment of such methods, wherein the dose of PD-1×CTLA-4 bispecific molecule administered in the maintenance period is different than the dose administered in the induction period.

The invention further provides the embodiment of such methods, wherein the PD-1×CTLA-4 bispecific molecule is administered by intravenous (IV) infusion.

The invention further provides the embodiment of such methods, wherein the IV infusion is over a period of between about 30 minutes to about 60 minutes.

The invention further provides the embodiment of such methods, wherein the cancer is selected from the group consisting of: an adrenal gland cancer, an AIDS-associated cancer, an alveolar soft part sarcoma, an astrocytic tumor, an anal cancer, a bile duct cancer, a bladder cancer, a bone cancer, a brain cancer, a brain and spinal cord cancer, a breast cancer, a HER2+ breast cancer, a triple negative breast cancer (TNBC), a carotid body tumors, a cervical cancer, an HPV-related cervical cancer, a cervical squamous cell carcinoma, a chondrosarcoma, a chordoma, a clear cell carcinoma, a colon cancer, a colorectal cancer (CRC), a microsatellite instability-high colorectal cancer (MSI-H CRC), a microsatellite-stable colorectal cancer (non-microsatellite-instability-high colorectal cancer, non-MSI-H CRC), a desmoplastic small round cell tumor, an endometrial cancer, an ependymoma, a Ewing's tumor, an extraskeletal myxoid chondrosarcoma, a fallopian tube carcinoma, a fibrogenesis imperfecta ossium, a fibrous dysplasia of the bone, a gallbladder or bile duct cancer, a gastric cancer, a gestational trophoblastic disease, a germ cell tumor, a glioblastoma, a head and neck cancer, an HPV-related head and neck cancer, a hematological malignancy, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a non-small-cell lung cancer (NSCLC), a medulloblastoma, a melanoma, a meningioma, Merkel cell carcinoma, a mesothelioma pharyngeal cancer, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer, a metastatic castration resistant prostate cancer (mCRPC), a posterior uveal melanoma, a renal cancer, a renal cell carcinoma (RCC), a rhabdoid tumor, a rhabdomyosarcoma, a sarcoma, a skin cancer, a small round blue cell tumor of childhood (including neuroblastoma and rhabdomyosarcoma), a soft-tissue sarcoma, a pleomorphic undifferentiated sarcoma, a dedifferentiated liposarcoma, a synovial sarcoma, a myxofibrosarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.

The invention further provides the embodiment of such methods, wherein the cancer is selected from the group consisting of: cervical cancer, HPV-related cervical cancer, cervical squamous cell carcinoma, CRC, MSI-H CRC, non-MSI-H CRC, head and neck cancer, HPV-related head and neck cancer, lung cancer, melanoma, NSCLC, prostate cancer, renal cancer, RCC, soft-tissue sarcoma, a pleomorphic undifferentiated sarcoma, a dedifferentiated liposarcoma, a synovial sarcoma, a myxofibrosarcoma, squamous cell cancer, and SCCHN.

The invention further provides the embodiment of such methods, wherein the cancer is cervical cancer. The invention particularly provides the embodiment of such methods, wherein the cervical cancer is cervical squamous cell carcinoma.

The invention further provides the embodiment of such methods, wherein the cancer is CRC. The invention particularly provides the embodiment of such methods, wherein the CRC is non-MSI-H CRC or is MSI-H CRC.

The invention further provides the embodiment of such methods, wherein the cancer is lung cancer. The invention particularly provides the embodiment of such methods, wherein the lung cancer is NSCLC.

The invention further provides the embodiment of such methods, wherein the cancer is melanoma. The invention particularly provides the embodiment of such methods, wherein the melanoma is cutaneous melanoma.

The invention further provides the embodiment of such methods, wherein the cancer is prostate cancer. The invention particularly provides the embodiment of such methods, wherein the prostate cancer is metastatic castration-resistant prostate cancer (mCRPC).

The invention further provides the embodiment of such methods, wherein the cancer is renal cancer. The invention particularly provides the embodiment of such methods, wherein the renal cancer is RCC.

The invention further provides the embodiment of such methods, wherein the cancer is soft tissue sarcoma. The invention particularly provides the embodiment of such methods, wherein the cancer is pleomorphic undifferentiated sarcoma, dedifferentiated liposarcoma, synovial sarcoma, or myxofibrosarcoma.

The invention further provides the embodiment of such methods, wherein the cancer is squamous cell cancer.

The invention further provides the embodiment of such methods, wherein the cancer is head and neck cancer.

The invention particularly provides the embodiment of such methods, wherein the squamous cell cancer or the head and neck cancer is SCCHN.

The invention further provides the embodiment of such methods, further comprising administering a therapeutically or prophylactically effective amount of one or more additional therapeutic agents or chemotherapeutic agents.

The invention further provides the embodiment of such methods, wherein the subject in need thereof is a human.

The invention provides a pharmaceutical kit comprising:
(a) a container comprising a PD-1×CTLA-4 bispecific molecule; and
(b) an instructional material, wherein the instructional material instructs that said PD-1×CTLA-4 bispecific molecule is to be used according to the method of any of the above embodiments.

The invention provides an embodiment for the use of such pharmaceutical kit according to such methods for the treatment of cancer.

The invention provides an embodiment for the use of such pharmaceutical kit according to such methods for stimulating immune cells.

The invention provides an embodiment for the use of such PD-1×CTLA-4 bispecific molecule according to such methods for the treatment of cancer.

The invention provides an embodiment for the use of such PD-1×CTLA-4 bispecific molecule according to such methods for stimulating immune cells.

### BRIEF DESCRIPTION OF THE DRAWINGS

FIG. 1 provides a schematic showing representative covalently bonded tetravalent diabody, such as a PD-1×CTLA-4 bispecific diabody, having four epitope-binding sites composed of two pairs of polypeptide chains (i.e., four polypeptide chains in all). One polypeptide of each pair has an E-coil Heterodimer-Promoting Domain and the other polypeptide of each pair has a K-coil Heterodimer-Promoting Domain. As shown, a cysteine residue may be present in a linker and/or in the Heterodimer-Promoting Domain. One polypeptide of each pair possesses a linker comprising a cysteine (which linker may comprise all or a portion of a hinge region) and CH2 and/or CH3 Domain, such that the associated chains form all or part of an Fc Region. VL and VH Domains that recognize the same epitope are shown using the same shading or fill pattern. The VL and VH Domains recognize different epitopes and the resulting molecule possesses four epitope-binding sites and is bispecific and bivalent with respect to each bound epitope.

FIGS. 2A-2C show the in vitro activity of a PD-1×CTLA-4 bispecific molecule. Representative experiments out of 3 or more independent repeats are shown. FIG. 2A shows the re-activation of beta-gal upon co-engagement of PD-1 and CTLA-4 by DART-D in a PathHunter® PD-1⁺CTLA-4⁺ assay. FIG. 2B shows the enhanced ability of DART-D to inhibit the binding of B7-1 to CTLA-4 (CTLA-4 blockade) on the surface of Jurkat PD-1⁺/CTLA-4⁺ cells as compared to its parental mAbs, their combination or isotype control. FIG. 2C shows blockade of B7-1 binding to Jurkat-PD-1⁺/CTLA-4⁺ by DART-D or CTLA-2 mAbs alone or in the presence of 10× concentration of competing PD-1 mAb demonstrating that the combination of DART-D in the presence of excess competing PD-1 mAb reduces the CTLA-4 blockade strength of DART-D due to lessening of avidity effect.

FIGS. 3A-3C show that a PD-1×CTLA-4 bispecific inhibitor enhances signaling and activation of T cells. Representative experiments out of 3 independent repeats are shown. FIG. 3A shows the results of a representative reporter assay, dual reporter cells and artificial APCs (Jurkat-PD-1⁺/CTLA-4⁺ and Raji-PD-L1⁺/B7⁺ cells, respectively) were co-cultured in the presence of DART-D, its parental PD-1 or CTLA-4 mAbs, their combination, replica of nivolumab, replica of ipilimumab, or their combination, and isotype control showing that DART-D rescues T-cell signaling. FIGS. 3B-3C, show the mean fold change of IL-2 concentrations relative to control IgG in SEB assay demonstrating that DART-D enhances T-cell activation, donors PBMC (N=39) were treated with the indicated concentrations of SEB in the presence of 10 ug/mL of DART-D, mAbs or control mAbs. IL-2 concentrations were normalized to levels observed in isotype control-treated samples. FIG. 3C shows a subset of donors (N=9/39) with reduced effects to PD-1 blockade (IL-2 f.c.<2) demonstrate enhanced responses to DART-D (the 25 ng SEB dose is shown).

FIGS. 4A-4G show that a PD-1×CTLA-4 bispecific molecule provides dual checkpoint blockade in vivo. Cynomolgus monkey (5F/5M) were infused with vehicle (●) 10 mg/kg/dose (▪), 40 mg/kg/dose (▴) or 100 mg/kg/dose (χ) DART-D at Day 1, 8, 15, and 22. DART-D serum concentrations, measured by ELISA (FIG. 4A), show that DART-D exhibited a linear PK with an antibody-like half-life of ˜7 days. Receptor occupancy, measured by flow cytometry (FIG. 4B), shows that binding to PD-1 correlated with its presence in the circulation. Error bars depict SEM, vertical dotted lines indicate dose administration, and the horizontal dotted line marks 100% cell surface binding. Splenocytes obtained 3 days after last infusion were stained for ICOS (FIG. 4C), showing a dose-dependent up regulation of ICOS on CD4⁺ T cells. Splenocytes were also analyzed for CD28/CD95 (co-) expression in CD4⁺ T cells, and CD25 or Ki67 expression in CD8⁺ T cells by flow cytometry. Fractions of cells expressing CD28 with low CD95 (naïve, FIG. 4D), CD28 and CD95 (memory, FIG. 4E), CD25⁺ (activated, FIG. 4F), or Ki67⁺ (proliferating, FIG. 4G) are plotted.

FIGS. 5A-5B display the treatment schemas for the study. Administration of DART-D is indicated by a filled star. Open stars indicate a continuation of Q3W dosing.

FIGS. 6A-6E show the pharmacokinetics and pharmacodynamics of DART-D in patients. FIG. 6A shows simulated multiple dose PK profiles for the 3, 6 and 10 mg/kg Q3W regimens with observed predose (open circles) and post-dose (closed circles) data superimposed, potential target concentration is overlaid as dashed line. FIG. 6B shows DART-D receptor occupancy for CD4⁺ T cells collected 43 days after second infusion (dose 3 pre-infusion, indicated by a “p”) compared to measured immediately after third infusion (dose 3 end of infusion (EOI), indicated by a “E”). Mean and SD are depicted. FIG. 6C shows the binding of DART-D-competing FACS mAbs to circulating T cells in patients treated with DART-D prior to first dose, 8 and 22 days later (first (●), second (▴) and third bar (▪), respectively at each dose level) (N=28). Bars indicate min to max interval. FIG. 6D shows upregulation of ICOS expression on peripheral blood CD4⁺ T cells measured before (●) and 8 days after (▪) first infusion of indicated doses of DART-D (N=28). FIG. 6E shows upregulation of ICOS expression (between day 1 and day 8) by circulating CD4⁺ T cells in patients treated with DART-D grouped by best overall response (PD—progressive disease; SD—stable disease; PR—partial response; CR—complete response; Unknown—not yet evaluable).

FIG. 7 presents a waterfall plot of the percent of change of target lesions (plotted as % change from baseline) among 13 response-evaluable cohort escalation patients treated with DART-D at doses ≥3 mg/kg, by tumor type and by dose. The dotted lines indicate a change from baseline of 20% or −30%. Abbreviations: CRC=colorectal carcinoma; EOC=epithelial ovarian cancer. “#” indicates previous treatment with a checkpoint inhibitor and “+” indicates patients staying on study at the time of data assembly.

### DETAILED DESCRIPTION OF THE INVENTION

The present invention in part is directed to dosing regimens for administering a PD-1×CTLA-4 bispecific molecule for the treatment of cancer and other diseases and conditions. The present invention also in part pertains to methods of using such PD-1×CTLA-4 bispecific molecules to stimulate immune cells. The invention in part concerns the use of such regimens for the administration of tetravalent PD-1×CTLA-4 bispecific diabodies that comprise two binding sites for PD-1 and two binding sites for CTLA-4. The invention in part is directed to the use of such bispecific molecules. The invention in part also is directed to the use of pharmaceutical compositions and pharmaceutical kits that contain such molecules, which facilitate the use of such dosing regimens in the treatment of cancer or to stimulate immune cells.

### I. PD-1×CTLA-4 Bispecific Molecules

A wide variety of recombinant bispecific antibody formats have been developed (see, e.g., WO 2008/003116, WO 2009/132876, WO 2008/003103, WO 2007/146968, WO 2009/018386, WO 2012/009544, and WO 2013/070565), most of which use linker peptides either to fuse a further epitope-binding fragment (e.g., an scFv, VL, VH, etc.) to, or within the antibody core (IgA, IgD, IgE, IgG or IgM), or to fuse multiple epitope-binding fragments (e.g., two Fab fragments or scFvs). Alternative formats use linker peptides to fuse an epitope-binding fragment (e.g., an scFv, VL, VH, etc.) to a dimerization domain such as the CH2-CH3 Domain or alternative polypeptides (WO 2005/070966, WO 2006/107786A WO 2006/107617A, and WO 2007/046893). WO 2013/174873, WO 2011/133886 and WO 2010/136172 disclose a trispecific antibody in which the CL and CH1 Domains are switched from their respective natural positions and the VL and VH Domains have been diversified (WO 2008/027236; WO 2010/108127) to allow them to bind to more than one antigen. WO 2013/163427 and WO 2013/119903 disclose modifying the CH2 Domain to contain a fusion protein adduct comprising a binding domain. WO 2010/028797, WO2010028796 and WO 2010/028795 disclose recombinant antibodies whose Fc Regions have been replaced with additional VL and VH Domains, so as to form trivalent binding molecules. WO 2003/025018 and WO2003012069 disclose recombinant diabodies whose individual chains contain scFv Domains. WO 2013/006544 discloses multivalent Fab molecules that are synthesized as a single polypeptide chain and then subjected to proteolysis to yield heterodimeric structures. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional binding domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another). Covalently bonding diabodies and trivalent molecules comprising diabody-like domains are described in WO 2015/184207, WO 2015/184203, WO 2012/162068; WO 2012/018687; WO 2010/080538; and WO 2006/113665, and are provided herein. Accordingly, it is specifically contemplated that the PD-1×CTLA-4 bispecific molecules of the present invention may have the structure of any of the above-described formats and may be produced any of the above-described methods.

A. Non-limiting Examples of PD-1 and CTLA-4 Binding Domains

In certain embodiments, the PD-1×CTLA-4 bispecific molecules of the present invention comprise:
(I) a PD-1-Binding Domain comprising a VL Domain (VL_{PD-1}) comprising PD-1-specific CDR_{L}1, CDR_{L}2 and CDR_{L}3 Domains, and a VH Domain (VH_{PD-1}) comprising PD-1-specific CDR_{H}1, CDR_{H}2 and CDR_{H}3 Domains; and
(II) a CTLA-4-Binding Domain comprising a VL Domain (VL_{CTLA-4}) comprising CTLA-4-specific CDR_{L}1, CDR_{L}2 and CDR_{L}3 Domains, and a VH Domain (VH_{CTLA-4}) comprising CTLA-4-specific CDR_{H}1, CDR_{H}2 and CDR_{H}3 Domains.

The amino acid sequence of a non-limiting example of a humanized VL_{PD-1} Domain is (SEQ ID NO:1):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI K
The Antigen Binding Domain of VL_{PD-1} comprises:
CDR_{L}1 SEQ ID NO: 2: RASESVDNYGMSFMN;
CDR_{L}2 SEQ ID NO: 3: AASNQGS;
and
CDR_{L}3 SEQ ID NO: 4: QQSKEVPYT.

The amino acid sequence of a non-limiting example of a humanized VH_{PD-1} Domain is (SEQ ID NO:5):

QVQLVQSGAE VKKPGASVKV SCKASGYSFT SYWMNWVRQA
PGQGLEWIGV IHPSDSETWL DQKFKDRVTI TVDKSTSTAY
MELSSLRSED TAVYYCAREH YGTSPFAYWG QGTLVTVSS
The Antigen Binding Domain of such VH_{PD-1} Domain
comprises:
CDR_{H}1 SEQ ID NO: 6: SYWMN;
CDR_{H}2 SEQ ID NO: 7: VIHPSDSETWLDQKFKD;
and
CDR_{H}3 SEQ ID NO: 8: EHYGTSPFAY.

The amino acid sequence of a non-limiting example of a humanized VL_{CTLA-4} Domain is (SEQ ID NO:9):

EIVLTQSPGT LSLSPGERAT LSCRASQSVS SSFLAWYQQK
PGQAPRLLIY GASSRATGIP DRESGSGSGT DFTLTISRLE
PEDFAVYYCQ QYGSSPWTFG QGTKVEIK
The Antigen Binding Domain of such VL_{CTLA-4} Domain
comprises:
CDR_{L}` SEQ ID NO: 10: RASQSVSSSFLA;
CDR_{L}2 SEQ ID NO: 11: GASSRAT;
and
CDR_{L}3 SEQ ID NO: 12: QQYGSSPWT

The amino acid sequence of a non-limiting example of a humanized VH_{CTLA-4} Domain is (SEQ ID NO:13):

QVQLVESGGG VVQPGRSLRL SCAASGFTFS SYTMHWVRQA
PGKGLEWVTF ISYDGSNKHY ADSVKGRFTV SRDNSKNTLY
LQMNSLRAED TAIYYCARTG WLGPFDYWGQ GTLVTVSS
The Antigen Binding Domain of such VH_{CTLA-4} Domain
comprises:
CDR_{H}1 SEQ ID NO: 14: SYTMH;
CDR_{H}2 SEQ ID NO: 15: FISYDGSNKHYADSVKG;
and
CDR_{H}3 SEQ ID NO: 16: TGWLGPFDY.

Alternative PD-1 binding domains may be used and numerous such domains have been described (see, for example, the amino acid sequences of: nivolumab (WHO Drug Information, 2013, Recommended INN: List 69, 27(1):68-69, INN No. 9623), pembrolizumab (WHO Drug Information, 2014, Recommended INN: List 75, 28(3):407, INN No. 9798), cemiplimab (WHO Drug Information, 2018, Proposed INN: List 119, 32(2):299, INN No. 10691), dostarlimab (WHO Drug Information 2018, Proposed INN: List 119, 32(2):307-308, INN No. 10787) and camrelizumab (WHO Drug Information, 2014, Recommended INN: List 77, 31(1):74, INN No. 10400)).

Alternative CTLA-4 binding domains may be used and numerous such domains have been described (see, for example, the amino acid sequences of: ipilimumab (WHO Drug Information, 2006, Recommended INN: List 56, 20(3):216, INN No. 8568; CAS No. 477202-00-9), tremelimumab (WHO Drug Information 2008, Recommended INN: List 59, 22(1):71, INN No. 8716; CAS No. 745013-59-6), nurulimab (WHO Drug Information 2019, Proposed INN: List 121, 33(2):302-303, INN No. 11141; CAS No. 2168561-20-2).

Amino acids from the Variable Domains of the mature heavy and light chains of immunoglobulins are designated by the position of an amino acid in the chain. Kabat described numerous amino acid sequences for antibodies, identified an amino acid consensus sequence for each subgroup, and assigned a residue number to each amino acid, and the CDRs are identified as defined by Kabat (Kabat et al., SEQUENCES OF PROTEINS OF IMMUNOLOGICAL INTEREST, 5^{th} Ed. Public Health Service, NH1, MD (1991); Martin, A. C. R. (1996) “Accessing the Kabat Antibody Sequence Database by Computer,” PROTEINS: Structure, Function and Genetics 25:130-133) (it will be understood that CDR_{H}1 as defined by Chothia, C. & Lesk, A. M. (1987) “Canonical Structures For The Hypervariable Regions Of Immunoglobulins,” J. Mol. Biol. 196:901-917) begins five residues earlier). Kabat's numbering scheme is extendible to antibodies not included in his compendium by aligning the antibody in question with one of the consensus sequences in Kabat by reference to conserved amino acids. This method for assigning residue numbers has become standard in the field and readily identifies amino acids at equivalent positions in different antibodies, including chimeric or humanized variants (see, e.g., Martin, A. C. R. (2010). “Chapter 3: Protein Sequence And Structure Analysis Of Antibody Variable Domains,” In: ANTIBODY ENGINEERING LAB MANUAL VOLUME 2 (2nd Edition) Duebel, S. and Kontermann, R. (Eds.) Springer-Verlag, Heidelberg). For example, an amino acid at position 50 of a human antibody light chain occupies the equivalent position to an amino acid at position 50 of a mouse antibody light chain.

B. Fc Receptor Binding Domains

In certain embodiments, the PD-1×CTLA-4 bispecific molecules of the present invention possess IgG CH2-CH3 Domains that are capable of complexing together to form an IgG Fc Receptor binding region (an “Fc Region”). The amino acid sequence of non-limiting examples of a CH2-CH3 domains of wild-type IgG1 (SEQ ID NO:24), IgG2 (SEQ ID NO:25), IgG3 (SEQ ID NO:26), and IgG4 (SEQ ID NO:27) are presented below.

The amino acid sequence of a non-limiting example of a CH2-CH3 domain of human IgG1 is (SEQ ID NO:24):

```
231 240 250 260 270 280
APELLGGPSV FLFPPKPKDT LMISRTPEVT CVVVDVSHED PEVKFNWYVD
              290 300 310 320 330
GVEVHNAKTK PREEQYNSTY RVVSVLTVLH QDWLNGKEYK CKVSNKALPA
              340 350 360 370 380
PIEKTISKAK GQPREPQVYT LPPSREEMTK NQVSLTCLVK GFYPSDIAVE
              390 400 410 420 430
WESNGQPENN YKTTPPVLDS DGSFFLYSKL TVDKSRWQQG NVFSCSVMHE
              440 447
ALHNHYTQKS LSLSPGX
```

wherein, X is a lysine (K) or is absent.

The amino acid sequence of the CH2-CH3 Domain of a non-limiting example of a human IgG2 is (SEQ ID NO:25):

```
231 240 250 260 270 280
APPVA-GPSV FLFPPKPKDT LMISRTPEVT CVVVDVSHED PEVQFNWYVD
              290 300 310 320 330
GVEVHNAKTK PREEQFNSTF RVVSVLTVVH QDWLNGKEYK CKVSNKGLPA
              340 350 360 370 380
PIEKTISKTK GQPREPQVYT LPPSREEMTK NQVSLTCLVK GFYPSDISVE
              390 400 410 420 430
WESNGQPENN YKTTPPMLDS DGSFFLYSKL TVDKSRWQQG NVFSCSVMHE
              440 447
ALHNHYTQKS LSLSPGX
```

wherein, X is a lysine (K) or is absent.

The amino acid sequence of the CH2-CH3 Domain of a non-limiting example of a human IgG3 is (SEQ ID NO:26):

```
231 240 250 260 270 280
APELLGGPSV FLFPPKPKDT LMISRTPEVT CVVVDVSHED PEVQFKWYVD
              290 300 310 320 330
GVEVHNAKTK PREEQYNSTF RVVSVLTVLH QDWLNGKEYK CKVSNKALPA
              340 350 360 370 380
PIEKTISKTK GQPREPQVYT LPPSREEMTK NQVSLTCLVK GFYPSDIAVE
              390 400 410 420 430
WESSGQPENN YNTTPPMLDS DGSFFLYSKL TVDKSRWQQG NIFSCSVMHE
              440 447
ALHNRFTQKS LSLSPGX
```

wherein, X is a lysine (K) or is absent.

The amino acid sequence of the CH2-CH3 Domain of a non-limiting example of a human IgG4 is (SEQ ID NO:27):

```
231 240 250 260 270 280
APELLGGPSV FLFPPKPKDT LMISRTPEVT CVVVDVSHED PEVQFKWYVD
              290 300 310 320 330
GVEVHNAKTK PREEQFNSTY RVVSVLTVLH QDWLNGKEYK CKVSNKGLPS
              340 350 360 370 380
SIEKTISKAK GQPREPQVYT LPPSQEEMTK NQVSLTCLVK GFYPSDIAVE
              390 400 410 420 430
WESSGQPENN YKTTPPVLDS DGSFFLYSRL TVDKSRWQEG NVFSCSVMHE
              440 447
ALHNHYTQKS LSLSLGX
```

wherein, X is a lysine (K) or is absent.

The numbering of the residues in the constant regions of an IgG heavy chain is that of the EU index as in Kabat et al., SEQUENCES OF PROTEINS OF IMMUNOLOGICAL INTEREST, 5^{th} Ed. Public Health Service, NH1, MD (1991), expressly incorporated herein by references. The “EU index as in Kabat” refers to the numbering of the human IgG1 EU antibody. Polymorphisms have been observed at a number of different positions within antibody constant regions (e.g., CH1 positions, including but not limited to positions 192, 193, and 214; Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the bispecific molecules of the present invention may incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the PD-1×CTLA-4 bispecific molecules of the invention. Specifically encompassed by the instant invention are DART-D molecules lacking the C-terminal residue of the CH3 Domain. Also specifically encompassed by the instant invention are such molecules comprising the C-terminal lysine residue of the CH3 Domain.

Although the Fc Region may possess the ability to bind to one or more Fc gamma receptor (FcγR), it is preferred that the Fc Regions of the PD-1×CTLA-4 bispecific molecules of the present invention will have been modified to have decreased (or substantially no) binding to one or more FcγR (e.g., FcγRIA (CD64), FcγRIIA (CD32A), FcγRIIB (CD32B), FcγRIIIA (CD16a) and/or FcγRIIIB (CD16b)) and/or reduced effector function relative to that exhibited by a wild-type Fc Region. Modifications that reduce or eliminate FcγR binding are well known in the art and include amino acid substitutions at positions 234 and 235, a substitution at position 265 or a substitution at position 297, wherein such numbering is that of the EU index as in Kabat (see, for example, U.S. Pat. No. 5,624,821, herein incorporated by reference). In one embodiment, the PD-1×CTLA-4 bispecific molecules of the present invention comprise a variant IgG1 Fc Region, wherein such variant IgG1 Fc Region comprises a substitution at position 234 with alanine and a substitution at position 235 with alanine (234A, 235A), wherein such numbering is that of the EU index as in Kabat. Alternatively, the Fc Region of the PD-1×CTLA-4 bispecific molecules of the present invention is one which inherently exhibits decreased (or substantially no) binding to one or more FcγR (particularly FcγRIIIA) and/or reduced effector function relative to that exhibited by a wild-type IgG1 Fc Region, such as an IgG2 or IgG4 Fc Region. In a specific embodiment, the PD-1×CTLA-4 bispecific molecules of the present invention comprise an IgG4 Fc Region.

Additionally, the serum half-life of molecules comprising an Fe Region may be increased by increasing the binding affinity of the Fc Region for FcRn. The term “half-life” as used herein means a pharmacokinetic property of a molecule that is a measure of the mean survival time of the molecules following their administration. Half-life can be expressed as the time required to eliminate fifty percent (50%) of a known quantity of the molecule from a subject's body (e.g., a human patient or other mammal) or a specific compartment thereof, for example, as measured in serum, i.e., circulating half-life, or in other tissues. In general, an increase in half-life results in an increase in mean residence time (MRT) in circulation for the molecule administered. Modifications capable of increasing the half-life of an Fc Region-containing molecule are known in the art and include, for example amino acid substitutions M252Y, S254T, T256E, and combinations thereof. For example, see the modifications described in U.S. Pat. Nos. 6,277,375, 7,083,784; 7,217,797, and 8,088,376; US2002/0147311 and US2007/0148164; and WO 98/23289; WO 2009/058492; and WO 2010/033279). In particular embodiments, the PD-1×CTLA-4 bispecific molecules of the present invention comprise a variant Fc Region, wherein such variant Fc Region comprises at least one amino acid modification relative to a wild-type Fc Region, such that such molecule has an increased half-life (relative to such a PD-1×CTLA-4 bispecific molecule having a wild-type Fc Region). In one embodiment, the PD-1×CTLA-4 bispecific molecules of the present invention comprise a variant Fc Region, wherein such variant Fc Region comprises a substitution at position 252 with tyrosine, 254 with threonine, and 256 with glutamate (252Y, 254T and 256E), wherein such numbering is that of the EU index as in Kabat.

In particular, the PD-1×CTLA-4 bispecific molecules of the present invention comprise a variant Fc Region, where such Fc Region comprises:
(A) one or more mutations which alter effector function and/or FcγR; and/or
(B) one or more mutations which extend serum half-life.

A non-limiting example of an IgG1 sequence for the CH2 and CH3 Domains of the PD-1×CTLA-4 bispecific molecules of the present invention will comprise the substitutions L234A/L235A/M252Y/S254T/T256E (SEQ ID NO:28):

APEAAGGPSV FLFPPKPKDT LYITREPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLTCLVK GFYPSDIAVE WESNGQPENN
YKTTPPVLDS DGSFFLYSKL TVDKSRWQQG NVFSCSVMHE
ALHNHYTQKS LSLSPGX

wherein, X is a lysine (K) or is absent.

A non-limiting example of an IgG4 sequence for the CH2 and CH3 Domains of the PD-1×CTLA-4 bispecific molecules of the present invention will comprise the M252Y/S254T/T256E substitutions (SEQ ID NO:29):

APEFLGGPSV FLFPPKPKDT LYITREPEVT CVVVDVSQED
PEVQFNWYVD GVEVHNAKTK PREEQFNSTY RVVSVLTVLH
QDWLNGKEYK CKVSNKGLPS SIEKTISKAK GQPREPQVYT
LPPSQEEMTK NQVSLTCLVK GFYPSDIAVE WESNGQPENN
YKTTPPVLDS DGSFFLYSRL TVDKSRWQEG NVFSCSVMHE
ALHNHYTQKS LSLSLGX

wherein, X is a lysine (K) or is absent.

C. PD-1×CTLA-4 Bispecific Diabodies

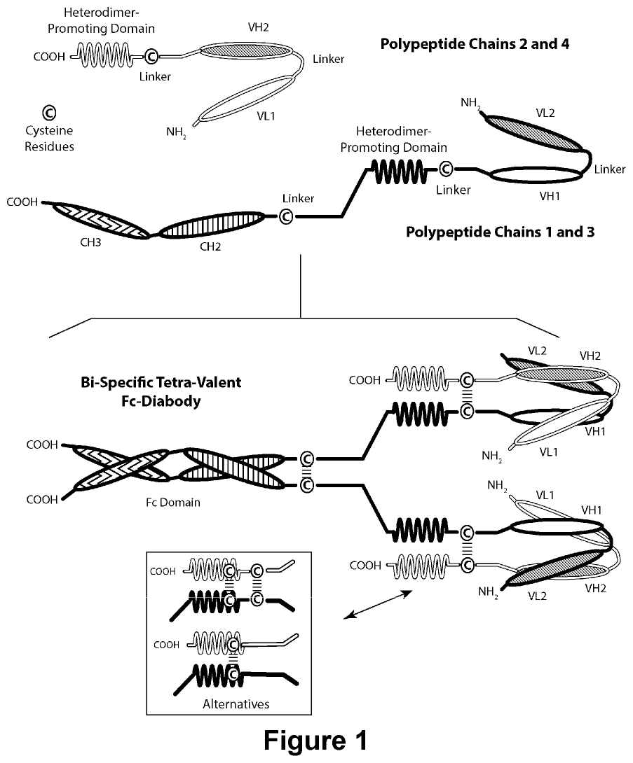

In certain embodiments, the PD-1×CTLA-4 bispecific molecules of the present invention are PD-1×CTLA-4 bispecific diabodies, preferably four chain, Fc Region-containing diabody having two binding sites specific for PD-1, two binding sites specific for CTLA-4, an Fc Region, and cysteine-containing E/K-coil Heterodimer-Promoting Domains. The general structure of such PD-1×CTLA-4 bispecific diabodies is provided in FIG. 1. Preferably such molecules comprise a VL and VH Domain of a humanized antibody that binds to PD-1 (VL_{PD-1} and VH_{PD-1}, respectively) and also a VL and VH Domain of a humanized antibody that binds to CTLA-4 (VL_{CTLA-4} and VH_{CTLA-4}, respectively). Thus, the PD-1×CTLA-4 bispecific diabodies of the invention are capable of specifically binding to an epitope of PD-1 and to an epitope of CTLA-4.

The PD-1×CTLA-4 bispecific diabodies of the present invention are engineered so that such first and second polypeptides covalently bond to one another via cysteine residues along their length. Such cysteine residues may be introduced into an intervening linker (Linker 1; e.g., GGGSGGGG (SEQ ID NO:17)), that separates the VL and VH Domains of the polypeptides. Alternatively, a second peptide that comprises a cysteine residue (Linker 2) is introduced into each polypeptide chain, for example, at a position N-terminal to the VL domain or C-terminal to the VH domain of such polypeptide chain. A non-limiting example of a sequence for such Linker 2 is SEQ ID NO:18: GGCGGG. Additionally or optionally, cysteine residues may be introduced into other domains, examples of which are provided below.

The formation of heterodimers can be further driven by engineering such polypeptide chains to contain Heterodimer-Promoting Domains, such as polypeptide coils of opposing charge. Thus, in one embodiment, one of the polypeptide chains will be engineered to contain an “E-coil” domain (SEQ ID NO:19: EVAALEK-EVAALEK-EVAALEK-EVAALEK) whose residues will form a negative charge at pH 7, while the other of the two polypeptide chains will be engineered to contain a “K-coil” domain (SEQ ID NO:20: KVAALKE-KVAALKE-KVAALKE-KVAALKE) whose residues will form a positive charge at pH 7. The presence of such charged domains promotes association between the first and second polypeptides, and thus fosters heterodimerization.

Alternatively, Heterodimer-Promoting Domains may be employed in which one of the four tandem “E-coil” helical domains of SEQ ID NO:19 has been modified to contain a cysteine residue (e.g., EVAACEK-EVAALEK-EVAALEK-EVAALEK (SEQ ID NO:21)), and/or in which one of the four tandem “K-coil” helical domains of SEQ ID NO:20 has been modified to contain a cysteine residue (e.g., KVAACKE-KVAALKE-KVAALKE-KVAALKE (SEQ ID NO:22)). Such embodiments are advantageously combined so that the Heterodimer-Promoting Domain(s) of SEQ ID NO:21 and the Heterodimer-Promoting Domain(s) of SEQ ID NO:22 are employed. An alternative, Linker 2 sequence lacking cysteine residues is SEQ ID NO:23: ASTKG, which may be employed with cysteine residue-containing Heterodimer-Promoting Domains.

It is immaterial which coil is provided to the first or second polypeptide chains. A non-limiting example of a PD-1×CTLA-4 bispecific diabody of the present invention, DART-D, has a first polypeptide chain having a E-coil sequence (e.g., SEQ ID NO:19 or SEQ ID NO:21) and a second polypeptide chain having a K-coil sequence (SEQ ID NO: 20 or SEQ ID NO:22).

The PD-1×CTLA-4 bispecific diabodies of the present invention may be engineered such that they possess IgG CH2-CH3 Domains that are capable of complexing together to form an Fc Region. In certain embodiments of the invention the PD-1×CTLA-4 bispecific diabodies of the present invention comprise human IgG CH2-CH3 Domains. A non-limiting example of human IgG CH2-CH3 Domains are provided above and a bispecific diabody of the invention can include CH2-CH3 Domains that have been engineered to modulate effector function and/or serum half-life.

In certain embodiments, the PD-1×CTLA-4 bispecific diabodies of the present invention are engineered with an intervening linker peptide (Linker 3) linking CH2 and CH3 Domains to the Heterodimer-Promoting Domains. Preferably Linker 3 is at a position C-terminal to the Heterodimer-Promoting Domain. Non-limiting examples of a Linker 3 that may be employed in the PD-1×CTLA-4 bispecific diabodies of the present invention include: GGGS (SEQ ID NO:30), LGGGSG (SEQ ID NO:31), ASTKG (SEQ ID NO:23), LEPKSS (SEQ ID NO:32), APSSS (SEQ ID NO:33), and APSSSPME (SEQ ID NO:34), GGC, and GGG. Linker 3 may comprise a portion of an IgG hinge region alone or in addition to other linker sequences. Non-limiting examples of hinge regions include: DKTHTCPPCP (SEQ ID NO:35) or EPKSCDKTHTCPPCP (SEQ ID NO:36) from IgG1, ERKCCVECPPCP (SEQ ID NO:37) from IgG2, ESKYGPPCPSCP (SEQ ID NO:38) from IgG4, and ESKYGPPCPPCP (SEQ ID NO:39) an IgG4 hinge variant comprising a stabilizing S228P substitution to reduce strand exchange ((Lu et al., (2008) “The Effect Of A Point Mutation On The Stability Of IgG4 As Monitored By Analytical Ultracentrifugation,” J. Pharmaceutical Sciences 97:960-969) to reduce the incidence of strand exchange). In certain embodiments, Linker 3 may further comprise GGG, for example GGGDKTHTCPPCP (SEQ ID NO:42).

D. DART-D

“DART-D” (also known as “MGD019”) is a non-limiting example of a PD-1×CTLA-4 bispecific molecule of the invention. DART-D is a bispecific, four chain, Fc Region-containing diabody having two binding sites specific for PD-1, two binding sites specific for CTLA-4, a variant IgG4 Fc Region engineered for extended half-life, and cysteine-containing E/K-coil Heterodimer-Promoting Domains. The four polypeptide chains that comprise DART-D are summarized in Table 1. The amino acid sequences are described in further detail below.

TABLE 1

| | Substituent Polypeptides (in the N- |
|---|---|
| DART-D | Terminal to C-Terminal Direction) |
| First and Third | SEQ ID NO: 1 |
| Polypeptide Chains | SEQ ID NO: 17 |
| (SEQ ID NO: 40) | SEQ ID NO: 13 |
| | SEQ ID NO: 18 |
| | SEQ ID NO: 21 |
| | SEQ ID NO: 39 |
| | SEQ ID NO: 29 |
| Second and Fourth | SEQ ID NO: 9 |
| Polypeptide Chains | SEQ ID NO: 17 |
| (SEQ ID NO: 41) | SEQ ID NO: 5 |
| | SEQ ID NO: 18 |
| | SEQ ID NO: 22 |

The first and third polypeptide chains of DART-D comprise, in the N-terminal to C-terminal direction: an N-terminus, a VL Domain of a monoclonal antibody capable of binding to PD-1 (VL_{PD-1}) (SEQ ID NO:1); an intervening linker peptide (Linker 1: GGGSGGGG (SEQ ID NO:17)); a VH Domain of a monoclonal antibody capable of binding to CTLA-4 (VH_{CTLA-4}) (SEQ ID NO:13); a cysteine-containing intervening linker peptide (Linker 2: GGCGGG (SEQ ID NO:18)); a cysteine-containing Heterodimer-Promoting (E-coil) Domain (EVAACEK-EVAALEK-EVAALEK-EVAALEK (SEQ ID NO:21)); an intervening linker peptide (Linker 3) comprising a stabilized IgG4 hinge region (SEQ ID NO:39); a variant IgG4 CH2-CH3 Domain comprising substitutions M252Y/S254T/T256E and lacking the C-terminal residue (SEQ ID NO:29); and a C-terminus.

The amino acid sequence of the first and third polypeptide chains of DART-D is (SEQ ID NO:40):

EIVLTQSPAT LSLSPGERAT LSCRASESVD NYGMSFMNWF
QQKPGQPPKL LIHAASNQGS GVPSRFSGSG SGTDFTLTIS
SLEPEDFAVY FCQQSKEVPY TFGGGTKVEI KGGGSGGGGQ
VQLVESGGGV VQPGRSLRLS CAASGFTFSS YTMHWVRQAP
GKGLEWVTFI SYDGSNKHYA DSVKGRFTVS RDNSKNTLYL
QMNSLRAEDT AIYYCARTGW LGPFDYWGQG TLVTVSSGGC
GGGEVAACEK EVAALEKEVA ALEKEVAALE KESKYGPPCP
PCPAPEFLGG PSVFLFPPKP KDTLYITREP EVTCVVVDVS
QEDPEVQFNW YVDGVEVHNA KTKPREEQFN STYRVVSVLT
VLHQDWLNGK EYKCKVSNKG LPSSIEKTIS KAKGQPREPQ
VYTLPPSQEE MTKNQVSLTC LVKGFYPSDI AVEWESNGQP
ENNYKTTPPV LDSDGSFFLY SRLTVDKSRW QEGNVFSCSV
MHEALHNHYT QKSLSLSLG

The second and fourth polypeptide chains of DART-D comprise, in the N-terminal to C-terminal direction: an N-terminus, a VL Domain of a monoclonal antibody capable of binding to CTLA-4 (VL_{CTLA-4}) (SEQ ID NO:9); an intervening linker peptide (Linker 1: GGGSGGGG (SEQ ID NO:17)); a VH Domain of a monoclonal antibody capable of binding PD-1 (VH_{PD-1}) (SEQ ID NO:5); a cysteine-containing intervening linker peptide (Linker 2: GGCGGG (SEQ ID NO:18)); a cysteine-containing Heterodimer-Promoting (K-coil) Domain (KVAACKE-KVAALKE-KVAALKE-KVAALKE (SEQ ID NO:22)); and a C-terminus.

The amino acid sequence of the second and fourth polypeptide chains of DART-D is (SEQ ID NO:41):

EIVLTQSPGT LSLSPGERAT LSCRASQSVS SSFLAWYQQK
PGQAPRLLIY GASSRATGIP DRFSGSGSGT DFTLTISRLE
PEDFAVYYCQ QYGSSPWTFG QGTKVEIKGG GSGGGGQVQL
VQSGAEVKKP GASVKVSCKA SGYSFTSYWM NWVRQAPGQG
LEWIGVIHPS DSETWLDQKF KDRVTITVDK STSTAYMELS
SLRSEDTAVY YCAREHYGTS PFAYWGQGTL VTVSSGGCGG
GKVAACKEKV AALKEKVAAL KEKVAALKE

Variants of DART-D may be readily generated by incorporating alternative VH/VL Domains, intervening linkers, Fc Regions, and/or by introducing one or more amino acid substitutions, additions or deletions. For example a variant IgG1 Fc Region engineered to reduce/abolish FcγR bindings and/or ADCC activity and for extended half-life is readily generated by incorporating CH2 and CH3 Domains comprising the substitutions L234A/L235A/M252Y/S254T/T256E (SEQ ID NO:28) instead of SEQ ID NO:29. Linker 3 of such variant may comprise an IgG1 hinge (SEQ ID NO:35, SEQ ID NO:36, or SEQ ID NO:42). Additional PD-1×CTLA-4 bispecific diabodies which may be used in the methods of the present invention are disclosed in WO 2017/019846 (see in particular “DART-B,” “DART-C,” “DART-E,” and “DART-F,” the sequences of which are described therein in Table 9, and are incorporated by reference herein).

E. Additional PD-1×CTLA-4 Bispecific Molecules

Other PD-1×CTLA-4 bispecific binding molecules which may be used in the method of the present invention are disclosed, for example, in: WO 2014/209804; WO 2017/218707; WO 2017/193032; WO 2019/094637; and US 2019/0185569.

Variants of such PD-1×CTLA-4 Bispecific Molecules may readily be generated, for example by incorporating alternative VH/VL Domains such as those provided herein.

### III. Methods of Production

The binding molecules of the invention (e.g., PD-1×CTLA-4 bispecific diabodies) can be may be made recombinantly and expressed using any method known in the art for the production of recombinant proteins. For example, nucleic acids encoding the polypeptide chains of such binding molecules can be constructed, introduced into an expression vector, and expressed in suitable host cells. The binding molecules may be recombinantly produced in bacterial cells (e.g., E. coli cells), or eukaryotic cells (e.g., CHO, 293E, COS, NS0 cells). In addition, the binding molecules can be expressed in a yeast cell such as Pichia, or Saccharomyces.

To produce the binding molecules (e.g., PD-1×CTLA-4 bispecific diabodies), one or more polynucleotides encoding the molecule may be constructed, introduced into an expression vector, and then expressed in suitable host cells. Standard molecular biology techniques are used to prepare the recombinant expression vector, transfect the host cells, select for transformants, culture the host cells and recover the molecules (See, for example, the techniques described in Green, M. R. et al., (2012), MOLECULAR CLONING, A LABORATORY MANUAL, 4th Ed., Cold Spring Harbor Laboratory, Cold Spring Harbor, NY and Ausubel et al. eds., 1998, CURRENT PROTOCOLS IN MOLECULAR BIOLOGY, John Wiley & Sons, NY). The expression vector(s) should have characteristics that permit replication of the vector in the host cell. The vector should also have promoter and signal sequences necessary for expression in the host cells. Such sequences are well known in the art. In addition to the nucleic acid sequence(s) encoding such binding molecules, the recombinant expression vectors may carry additional sequences, such as sequences that regulate replication of the vector in host cells (e.g., origins of replication) and selectable marker genes. Another method that may be employed is to express the gene sequence in plants (e.g., tobacco) or a transgenic animal. Suitable methods useful for expressing such binding molecules recombinantly in plants or milk have been disclosed (see, for example, Peeters et al. (2001) “Production Of Antibodies And Antibody Fragments In Plants,” Vaccine 19:2756; U.S. Pat. No. 5,849,992; and Pollock et al. (1999) “Transgenic Milk As A Method For The Production Of Recombinant Antibodies,” J. Immunol Methods 231:147-157).

Once a binding molecule has been recombinantly expressed, it may be purified from inside or outside (such as from culture media) of the host cell by any method known in the art for purification of polypeptides or polyproteins. Methods for isolation and purification commonly used for antibody purification (e.g., antibody purification schemes based on antigen selectivity) may be used for the isolation and purification of such molecules and are not limited to any particular method. For example, one or more of the following methods may be used: column chromatography, filtration, ultrafiltration, salting out, solvent precipitation, solvent extraction, distillation, immunoprecipitation, SDS-polyacrylamide gel electrophoresis, isoelectric focusing, dialysis, and recrystallization. Chromatography includes, e.g., ion exchange, affinity, particularly by affinity for the specific antigen (optionally after Protein A selection where the PD-1×CTLA-4 bispecific molecule comprises an Fc Region), sizing column chromatography, hydrophobic, gel filtration, reverse-phase, and adsorption (Marshak et al. (1996) STRATEGIES FOR PROTEIN PURIFICATION AND CHARACTERIZATION: A Laboratory Course Manual. (Eds.), Cold Spring Harbor Laboratory Press, Cold Spring Harbor, NY).

### IV. Uses of a PD-1×CTLA-4 Bispecific Molecule of the Invention

The PD-1×CTLA-4 bispecific molecules of the present invention generally have the ability to inhibit PD-1 and CTLA-4 function and thus augment the immune system by blocking immune system inhibition mediated by PD-1 and CTLA-4. The PD-1×CTLA-4 bispecific molecules of the present invention also generally allow for full blockade of both PD-1 and CTLA-4, as well as blockade that is biased toward CTLA-4 when co-expressed with PD-1. Thus, the PD-1×CTLA-4 bispecific molecules of the invention generally are useful for relieving T-cell exhaustion and/or augmenting an immune response (e.g., a T-cell and/or NK-cell mediated immune response) of a subject. In particular, the PD-1×CTLA-4 bispecific molecules of the invention may be used to treat any disease or condition associated with an undesirably suppressed immune system, including cancer. As used herein, the term “subject” refers to a human (i.e., a human patient) or other mammal. Non-limiting examples of dosing regimens for administering such therapy to a subject in need thereof are provided herein.

The cancers that may be treated with a PD-1×CTLA-4 bispecific molecule of the present invention include: an adrenal gland cancer, an AIDS-associated cancer, an alveolar soft part sarcoma, an astrocytic tumor, an anal cancer, a bile duct cancer, a bladder cancer, a bone cancer, a brain cancer, a brain and spinal cord cancer, a breast cancer, a HER2+ breast cancer, a triple negative breast cancer (TNBC), a carotid body tumors, a cervical cancer, an HPV-related cervical cancer, a cervical squamous cell carcinoma, a chondrosarcoma, a chordoma, a clear cell carcinoma, a colon cancer, a colorectal cancer (CRC), a microsatellite instability-high colorectal cancer (MSI-H CRC), a microsatellite-stable colorectal cancer (non-microsatellite-instability-high colorectal cancer, non-MSI-H CRC), a desmoplastic small round cell tumor, an endometrial cancer, an ependymoma, a Ewing's tumor, an extraskeletal myxoid chondrosarcoma, a fallopian tube carcinoma, a fibrogenesis imperfecta ossium, a fibrous dysplasia of the bone, a gallbladder or bile duct cancer, a gastric cancer, a gestational trophoblastic disease, a germ cell tumor, a glioblastoma, a head and neck cancer, an HPV-related head and neck cancer, a hematological malignancy, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a non-small-cell lung cancer (NSCLC), a medulloblastoma, a melanoma, a meningioma, Merkel cell carcinoma, a mesothelioma pharyngeal cancer, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer, a metastatic castration resistant prostate cancer (mCRPC), a posterior uveal melanoma, a renal cancer, a renal cell carcinoma (RCC), a rhabdoid tumor, a rhabdomyosarcoma, a sarcoma, a skin cancer, a small round blue cell tumor of childhood (including neuroblastoma and rhabdomyosarcoma), a soft-tissue sarcoma, a pleomorphic undifferentiated sarcoma, a dedifferentiated liposarcoma, a synovial sarcoma, a myxofibrosarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.

In particular, a PD-1×CTLA-4 bispecific molecule of the present invention may be used in the treatment of: cervical cancer, HPV-related cervical cancer, cervical squamous cell carcinoma, CRC, MSI-H CRC, non-MSI-H CRC, head and neck cancer, HPV-related head and neck cancer, lung cancer, melanoma, NSCLC, prostate cancer, renal cancer, RCC, soft-tissue sarcoma, a pleomorphic undifferentiated sarcoma, a dedifferentiated liposarcoma, a synovial sarcoma, a myxofibrosarcoma, squamous cell cancer, and SCCHN.

In certain embodiments, a PD-1×CTLA-4 bispecific molecule of the present invention is administered as a first-line therapy for treatment of cancer. In certain embodiments, a PD-1×CTLA-4 bispecific molecule of the present invention is administered after one or more prior lines of therapy. In certain embodiments, a PD-1×CTLA-4 bispecific molecule of the present invention can be employed as an adjuvant therapy at the time of, or after surgical removal of a tumor in order to delay, suppress or prevent the development of metastasis. A PD-1×CTLA-4 bispecific molecule of the present invention can also be administered before surgery (e.g., as a neoadjuvant therapy) in order to decrease the size of the tumor and thus enable or simplify such surgery, spare tissue during such surgery, and/or decrease any resulting disfigurement.

The invention specifically encompasses administering a PD-1×CTLA-4 bispecific molecule in combination with a therapeutically or prophylactically effective amount of one or more other agents or therapies known to those skilled in the art for the treatment and/or prevention of cancer, including but not limited to, current standard and experimental chemotherapeutic agents or chemotherapies, hormonal agents or therapies, biological agents or therapies, immunotherapeutic agents or immunotherapies, radiation agents or therapies, other therapeutic agents, or surgery.

As used herein, the term “combination” refers to the use of more than one therapeutic agent. The use of the term “combination” does not restrict the order in which therapeutic agents are administered to a subject (e.g., a human patient or other mammal) with a disorder, nor does it mean that the agents are administered at exactly the same time. The term combination means that a PD-1×CTLA-4 bispecific molecule of the present invention, and any other therapeutic or chemotherapeutic agent, are administered to a human patient, or other mammal, in a sequence and within a time interval such that the combination of a PD-1×CTLA-4 bispecific molecule and the other agent provide an increased benefit than if they were administered otherwise. For example, each therapeutic therapy (e.g., chemotherapy, radiation therapy, hormonal therapy or biological therapy) may be administered at the same time or sequentially, in any order, at different points in time; however, if not administered at the same time, they should be administered sufficiently close in time so as to provide the desired therapeutic or prophylactic effect. Each therapeutic agent can be administered separately, independently in any appropriate form and independently by any suitable route, e.g., one by the oral route and one parenterally, etc.

### V. Methods of Administration and Dose

A PD-1×CTLA-4 bispecific molecule of the invention can be administered by a variety of methods to a subject, e.g., a subject in need thereof, for example a human patient. For many applications, the route of administration is one of: intravenous injection or infusion (IV), subcutaneous injection (SC), intraperitoneal injection (IP), or intramuscular injection. It is also possible to use intra-articular delivery. Other modes of parenteral administration can also be used. Non-limiting examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection.

The PD-1×CTLA-4 bispecific molecule may be administered using a weight-based dose. The dose can be selected to reduce or avoid production of antibodies against the administered molecules. Dosage regimens are adjusted to provide the desired response, e.g., a therapeutic response or a combinatorial therapeutic effect. Generally, doses of a PD-1×CTLA-4 bispecific molecule (and optionally a further agent) can be used in order to provide a subject with the agent in bioavailable quantities. As used herein, the term “dose” refers to a specified amount of medication taken at one time. The term “dosage” refers to the administering of a specific amount, number, and frequency of doses over a specified period of time; the term dosage thus includes chronological features, such as duration and periodicity.

The term “weight-based dose” as used herein, refers to a discrete amount of a molecule to be administered per a unit of weight of a subject, for example milligrams of drug per kilograms of a subject's body weight (mg/kg body weight, abbreviated herein as “mg/kg”). The calculated dose will be administered based on the subject's body weight at baseline. Typically, a significant (≥10%) change in body weight from baseline or established plateau weight will generally prompt recalculation of dose. Single or multiple dosages may be given. Compositions comprising a PD-1×CTLA-4 bispecific molecule may be administered to a subject in need thereof via infusion.

In certain embodiments, a PD-1×CTLA-4 bispecific molecule is administered to a subject in need thereof at a weight-based dose of about 3 mg/kg to about 10 mg/kg, about 3 mg/kg to about 8 mg/kg, about 3 mg/kg to about 6 mg/kg, about 6 mg/kg to about 10 mg/kg, about 6 mg/kg to about 9 mg/kg, about 6 mg/kg to about 8 mg/kg, about 6 mg/kg to about 7 mg/kg, about 7 mg/kg to about 8 mg/kg, about 8 mg/kg to about 9 mg/kg, or about 9 mg/kg to about 10 mg/kg. In specific embodiments, a PD-1×CTLA-4 bispecific molecule is administered to a subject in need thereof at a weight-based dose of about 3 mg/kg, about 4 mg/kg, about 5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In certain embodiments, a PD-1×CTLA-4 bispecific molecule is to be administered at any one of the foregoing doses at a dosage of about once every 3 weeks to about once every 6 weeks (e.g., about once every 4 weeks, about once every 5 weeks) during a treatment. In certain embodiments, a PD-1×CTLA-4 bispecific molecule is to be administered at a first dose at a first dosage one or more times and at a second dose at a second dosage one or more times, where the first dose and the second dose are the same or different and the first dosage and the second dosage are the same of different. In some embodiments, the first dose and the second dose are the same (e.g., about 6 mg/kg) and the first dosage and the second dosage are the same (e.g., about once every 3 weeks). In some embodiments, the first dose and the second dose are the same (e.g., about 6 mg/kg) and the first dosage and the second dosage are different (e.g., first dosage is about once every 3 weeks and second dosage is about once every 6 weeks). In some embodiments, the first dose and the second dose are different (e.g., first dose at about 6 mg/kg and second dose at about 3 mg/kg) and the first dosage and the second dosage are the same (e.g., about once every 3 weeks). In some embodiments, the first dose and the second dose are different (e.g., first dose at about 6 mg/kg and second dose at about 3 mg/kg) and the first dosage and the second dosage are different (e.g., first dosage is about once every 3 weeks and second dosage is about once every 6 weeks).

With respect to weight-based doses, the term “about” is intended to denote a range that is 10% greater than a recited dose or 10% less than a recited dose, such that for example, a dose of about 10 mg/kg will be between 9 mg/kg and 11 mg/kg.

The terms “dosing interval” and “dosing intervals” as used herein, refer to the time interval between doses, which can be regular or intermittent. A dosage of a PD-1×CTLA-4 bispecific molecule can be administered at periodic dosing intervals over a period of time sufficient to encompass at least 2 doses, at least 4 doses, at least 6 doses, at least 12 doses, or at least 22 doses (a course of treatment), for example. For example, a dosage may be administered e.g., once or twice daily, or about one to four times per week, or particularly once every week (“Q1W”), once every two weeks (“Q2W”), once every three weeks (“Q3W”), once every four weeks (“Q4W”), once every six weeks (“Q6W”), and the like. Such periodic administration may continue for a period of time e.g., for between about 1 week to 52 weeks, for 24 weeks, for more than 52 weeks, for 84 weeks, or for more than 84 weeks. Such course of treatment may be divided into increments, each referred to herein as a “cycle,” of e.g., between 2 weeks to 12 weeks, between about 3 weeks to 12 weeks, particularly about 4 weeks, or about 6 weeks, or about 12 weeks, during which a set number of doses are administered. Such periodic administration may continue for a period of time e.g., for between about 7 days to 364 days, for 168 days, for more than 364 days, or for 588 days. Such course of treatment may be divided into increments, each referred to herein as a “cycle,” of e.g., between 14 days to 84 days, between about 21 days to 84 days, particularly about 28 days, or about 42 days, or about 84 days, during which a set number of doses are administered. The dose and/or the frequency of administration may be the same or different during each cycle. Factors that may influence the dosage and timing required to effectively treat a subject, include, e.g., the severity of the disease or disorder, formulation, route of delivery, previous treatments, the general health and/or age of the subject, and the presence of other diseases in the subject. Moreover, treatment of a subject with a therapeutically effective amount of a compound can include a single treatment or, can include a series of treatments. A treatment may include one or more periods during which the dose administered and/or frequency of such administration may be the same or different.

In one embodiment, a PD-1×CTLA-4 bispecific molecule is administered at a specified dose and dosing interval during an induction period, and the PD-1×CTLA-4 bispecific molecule is administered at a specified dose and dosing interval during a subsequent maintenance period. In certain embodiments, the dose administered during a maintenance period is the same as the dose administered during an induction period. In certain embodiments, the dose administered during a maintenance period is different from the dose administered during an induction period. In certain embodiments, the dosing interval during a maintenance period is different from the dosing interval during an induction period. In certain embodiments, the dosing interval during a maintenance period is the same as the dosing interval during an induction period. In a specific embodiment, the dose administered during a maintenance period is the same as the dose administered during an induction period, and the dosing interval during a maintenance period is different from the dosing interval during an induction period. In a specific embodiment, the dose administered during a maintenance period is the same as the dose administered during an induction period, and the dosing interval during a maintenance period is the same as the dosing interval during an induction period (i.e., the dose administered and dosing interval are unchanged during the course of treatment). In certain embodiments, an induction period is about 24 weeks. In certain embodiments, an induction period is about 168 days. In certain embodiments, about 8 doses of a PD-1×CTLA-4 bispecific molecule are administered during an induction period. In certain embodiments, a maintenance period is between about 6 weeks to about 84 weeks. In certain embodiments, a maintenance period is between 7 days and 588 days. In certain embodiments, the treatment period is at least about 24 week, at least about 36 weeks, at least about 48 weeks, at least about 60 weeks, at least about 72 weeks, at least about 84 weeks, or more than 84 weeks. In certain embodiments at least one dose of a PD-1×CTLA-4 bispecific molecule is administered during a maintenance period, and additional doses may be administered until remission of disease or unmanageable toxicity is observed. In certain embodiments, treatment continues for a period of time after remission of disease. In a specific embodiment, at least one dose of a PD-1×CTLA-4 bispecific molecule is administered during a maintenance period, and additional doses may be administered until about 14 doses have been administered. In a specific embodiment, at least one dose of a PD-1×CTLA-4 bispecific molecule is administered during a maintenance period, and additional doses may be administered until about 28 doses have been administered.

A “dosing regimen” is a dosage administration in which a subject is administered a predetermined dose (or set of such predetermined doses) at a predetermined frequency (or set of such frequencies) for a predetermined periodicity (or periodicities). A non-limiting example of a dosing regimen comprises administration of a PD-1×CTLA-4 bispecific molecule of the present invention at a weight-based dose of about 3 mg/kg to about 10 mg/kg once every 3 weeks during an induction period. Another non-limiting example of a dosing regimen comprises administration of a PD-1×CTLA-4 bispecific molecule of the present invention at a weight-based dose of about 3 mg/kg to about 8 mg/kg once every 3 weeks during an induction period. Another non-limiting example of a dosing regimen comprises administration of a PD-1×CTLA-4 bispecific molecule of the present invention at a weight-based dose of about 3 mg/kg to about 6 mg/kg once every 3 weeks during an induction period. Another non-limiting example of a dosing regimen comprises administration of a PD-1×CTLA-4 bispecific molecule at a weight-based dose of about 6 mg/kg to about 10 mg/kg once every 3 weeks during an induction period. Another non-limiting example of a dosing regimen comprises administration of a PD-1×CTLA-4 bispecific molecule at a weight-based dose of about 6 mg/kg to about 9 mg/kg once every 3 weeks during an induction period. Another non-limiting example of a dosing regimen comprises administration of a PD-1×CTLA-4 bispecific molecule at a weight-based dose of about 6 mg/kg to about 8 mg/kg once every 3 weeks during an induction period. Another non-limiting example of a dosing regimen comprises administration of a PD-1×CTLA-4 bispecific molecule at a weight-based dose of about 6 mg/kg to about 7 mg/kg once every 3 weeks during an induction period. Another non-limiting example of a dosing regimen comprises administration of a PD-1×CTLA-4 bispecific molecule at a weight-based dose of about 7 mg/kg to about 8 mg/kg once every 3 weeks during an induction period. Another non-limiting example of a dosing regimen comprises administration of a PD-1×CTLA-4 bispecific molecule at a weight-based dose of about 8 mg/kg to about 9 mg/kg once every 3 weeks during an induction period. Another non-limiting example of a dosing regimen comprises administration of a PD-1×CTLA-4 bispecific molecule at a weight-based dose of about 9 mg/kg to about 10 mg/kg once every 3 weeks during an induction period. Another non-limiting example of a dosing regimen comprises administration of a PD-1×CTLA-4 bispecific molecule at a weight-based dose of about 3 mg/kg once every 3 weeks during an induction period. Another non-limiting example of a dosing regimen comprises administration of a PD-1×CTLA-4 bispecific molecule at a weight-based dose of about 4 mg/kg once every 3 weeks during an induction period. Another non-limiting example of a dosing regimen comprises administration of a PD-1×CTLA-4 bispecific molecule at a weight-based dose of about 5 mg/kg once every 3 weeks during an induction period. Another non-limiting example of a dosing regimen comprises administration of a PD-1×CTLA-4 bispecific molecule at a weight-based dose of about 6 mg/kg once every 3 weeks during an induction period. Another non-limiting example of a dosing regimen comprises administration of a PD-1×CTLA-4 bispecific molecule at a weight-based dose of about 6.5 mg/kg once every 3 weeks during an induction period. Another non-limiting example of a dosing regimen comprises administration of a PD-1×CTLA-4 bispecific molecule at a weight-based dose of about 7 mg/kg once every 3 weeks during an induction period. Another non-limiting example of a dosing regimen comprises administration of a PD-1×CTLA-4 bispecific molecule at a weight-based dose of about 7.5 mg/kg once every 3 weeks during an induction period. Another non-limiting example of a dosing regimen comprises administration of a PD-1×CTLA-4 bispecific molecule at a weight-based dose of about 8 mg/kg once every 3 weeks during an induction period. Another non-limiting example of a dosing regimen comprises administration of a PD-1×CTLA-4 bispecific molecule at a weight-based dose of about 8.5 mg/kg once every 3 weeks during an induction period. Another non-limiting example of a dosing regimen comprises administration of a PD-1×CTLA-4 bispecific molecule at a weight-based dose of about 9 mg/kg once every 3 weeks during an induction period. Another non-limiting example of a dosing regimen comprises administration of a PD-1×CTLA-4 bispecific molecule at a weight-based dose of about 9.5 mg/kg once every 3 weeks during an induction period. Another non-limiting example of a dosing regimen comprises administration of a PD-1×CTLA-4 bispecific molecule at a weight-based dose of about 10 mg/kg once every 3 weeks during an induction period.

A non-limiting example of a dosing regimen comprises administration of a PD-1×CTLA-4 bispecific molecule of the present invention at a weight-based dose of about 3 mg/kg to about 10 mg/kg once every 6 weeks during a maintenance period. Another non-limiting example of a dosing regimen comprises administration of a PD-1×CTLA-4 bispecific molecule of the present invention at a weight-based dose of about 3 mg/kg to about 8 mg/kg once every 6 weeks during a maintenance period. Another non-limiting example of a dosing regimen comprises administration of a PD-1×CTLA-4 bispecific molecule of the present invention at a weight-based dose of about 3 mg/kg to about 6 mg/kg once every 6 weeks during a maintenance period. Another non-limiting example of a dosing regimen comprises administration of a PD-1×CTLA-4 bispecific molecule at a weight-based dose of about 6 mg/kg to about 10 mg/kg once every 6 weeks during a maintenance period. Another non-limiting example of a dosing regimen comprises administration of a PD-1×CTLA-4 bispecific molecule at a weight-based dose of about 6 mg/kg to about 9 mg/kg once every 6 weeks during a maintenance period. Another non-limiting example of a dosing regimen comprises administration of a PD-1×CTLA-4 bispecific molecule at a weight-based dose of about 6 mg/kg to about 8 mg/kg once every 6 weeks during a maintenance period. Another non-limiting example of a dosing regimen comprises administration of a PD-1×CTLA-4 bispecific molecule at a weight-based dose of about 6 mg/kg to about 7 mg/kg once every 6 weeks during a maintenance period. Another non-limiting example of a dosing regimen comprises administration of a PD-1×CTLA-4 bispecific molecule at a weight-based dose of about 7 mg/kg to about 8 mg/kg once every 6 weeks during a maintenance period. Another non-limiting example of a dosing regimen comprises administration of a PD-1×CTLA-4 bispecific molecule at a weight-based dose of about 8 mg/kg to about 9 mg/kg once every 6 weeks during a maintenance period. Another non-limiting example of a dosing regimen comprises administration of a PD-1×CTLA-4 bispecific molecule at a weight-based dose of about 9 mg/kg to about 10 mg/kg once every 6 weeks during a maintenance period. Another non-limiting example of a dosing regimen comprises administration of a PD-1×CTLA-4 bispecific molecule at a weight-based dose of about 3 mg/kg once every 6 weeks during a maintenance period. Another non-limiting example of a dosing regimen comprises administration of a PD-1×CTLA-4 bispecific molecule at a weight-based dose of about 4 mg/kg once every 6 weeks during a maintenance period. Another non-limiting example of a dosing regimen comprises administration of a PD-1×CTLA-4 bispecific molecule at a weight-based dose of about 5 mg/kg once every 6 weeks during a maintenance period. Another non-limiting example of a dosing regimen comprises administration of a PD-1×CTLA-4 bispecific molecule at a weight-based dose of about 6 mg/kg once every 6 weeks during a maintenance period. Another non-limiting example of a dosing regimen comprises administration of a PD-1×CTLA-4 bispecific molecule at a weight-based dose of about 6.5 mg/kg once every 6 weeks during a maintenance period. Another non-limiting example of a dosing regimen comprises administration of a PD-1×CTLA-4 bispecific molecule at a weight-based dose of about 7 mg/kg once every 6 weeks during a maintenance period. Another non-limiting example of a dosing regimen comprises administration of a PD-1×CTLA-4 bispecific molecule at a weight-based dose of about 7.5 mg/kg once every 6 weeks during a maintenance period. Another non-limiting example of a dosing regimen comprises administration of a PD-1×CTLA-4 bispecific molecule at a weight-based dose of about 8 mg/kg once every 6 weeks during a maintenance period. Another non-limiting example of a dosing regimen comprises administration of a PD-1×CTLA-4 bispecific molecule at a weight-based dose of about 8.5 mg/kg once every 6 weeks during a maintenance period. Another non-limiting example of a dosing regimen comprises administration of a PD-1×CTLA-4 bispecific molecule at a weight-based dose of about 9 mg/kg once every 6 weeks during a maintenance period. Another non-limiting example of a dosing regimen comprises administration of a PD-1×CTLA-4 bispecific molecule at a weight-based dose of about 9.5 mg/kg once every 6 weeks during a maintenance period. Another non-limiting example of a dosing regimen comprises administration of a PD-1×CTLA-4 bispecific molecule at a weight-based dose of about 10 mg/kg once every 6 weeks during a maintenance period.

A non-limiting example of a dosing regimen comprises administration of a PD-1×CTLA-4 bispecific molecule of the present invention at a weight-based dose of about 3 mg/kg to about 10 mg/kg once every 3 weeks during an induction period, and at the same dose once every 6 weeks during a maintenance period. Another non-limiting example of a dosing regimen comprises administration of a PD-1×CTLA-4 bispecific molecule of the present invention at a weight-based dose of about 3 mg/kg to about 8 mg/kg once every 3 weeks during an induction period, and at the same dose once every 6 weeks during a maintenance period. Another non-limiting example of a dosing regimen comprises administration of a PD-1×CTLA-4 bispecific molecule of the present invention at a weight-based dose of about 3 mg/kg to about 6 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 6 mg/kg to about 10 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 6 mg/kg to about 9 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 6 mg/kg to about 8 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 6 mg/kg to about 7 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 7 mg/kg to about 8 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 8 mg/kg to about 9 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 9 mg/kg to about 10 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 3 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 4 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 5 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 6 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 6.5 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 7 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 7.5 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 8 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 8.5 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 9 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 9.5 mg/kg once every 3 weeks during an induction period, and at the same dose once every 6 weeks during a maintenance period. Another non-limiting example of a dosing regimen comprises administration of a PD-1×CTLA-4 bispecific molecule at a weight-based dose of about 10 mg/kg once every 3 weeks during an induction period, and at the same dose once every 6 weeks during a maintenance period.

As provided above, in certain embodiments the dose administered and dosing interval are unchanged during the course of treatment. A non-limiting example of such a dosing regimen comprises administration of a PD-1×CTLA-4 bispecific molecule of the present invention at a weight-based dose of about 3 mg/kg to about 10 mg/kg once every 3 weeks for the duration of treatment. Another non-limiting example of a dosing regimen comprises administration of a PD-1×CTLA-4 bispecific molecule of the present invention at a weight-based dose of about 3 mg/kg to about 8 mg/kg once every 3 weeks for the duration of treatment. Another non-limiting example of a dosing regimen comprises administration of a PD-1×CTLA-4 bispecific molecule of the present invention at a weight-based dose of about 3 mg/kg to about 6 mg/kg once every 3 weeks for the duration of treatment. Another non-limiting example of a dosing regimen comprises administration of a PD-1×CTLA-4 bispecific molecule at a weight-based dose of about 6 mg/kg to about 10 mg/kg once every 3 weeks for the duration of treatment. Another non-limiting example of a dosing regimen comprises administration of a PD-1×CTLA-4 bispecific molecule at a weight-based dose of about 6 mg/kg to about 9 mg/kg once every 3 weeks for the duration of treatment. Another non-limiting example of a dosing regimen comprises administration of a PD-1×CTLA-4 bispecific molecule at a weight-based dose of about 6 mg/kg to about 8 mg/kg once every 3 weeks for the duration of treatment. Another non-limiting example of a dosing regimen comprises administration of a PD-1×CTLA-4 bispecific molecule at a weight-based dose of about 6 mg/kg to about 7 mg/kg once every 3 weeks for the duration of treatment. Another non-limiting example of a dosing regimen comprises administration of a PD-1×CTLA-4 bispecific molecule at a weight-based dose of about 7 mg/kg to about 8 mg/kg once every 3 weeks for the duration of treatment. Another non-limiting example of a dosing regimen comprises administration of a PD-1×CTLA-4 bispecific molecule at a weight-based dose of about 8 mg/kg to about 9 mg/kg once every 3 weeks for the duration of treatment. Another non-limiting example of a dosing regimen comprises administration of a PD-1×CTLA-4 bispecific molecule at a weight-based dose of about 9 mg/kg to about 10 mg/kg once every 3 weeks for the duration of treatment. Another non-limiting example of a dosing regimen comprises administration of a PD-1×CTLA-4 bispecific molecule at a weight-based dose of about 3 mg/kg once every 3 weeks for the duration of treatment. Another non-limiting example of a dosing regimen comprises administration of a PD-1×CTLA-4 bispecific molecule at a weight-based dose of about 4 mg/kg once every 3 weeks for the duration of treatment. Another non-limiting example of a dosing regimen comprises administration of a PD-1×CTLA-4 bispecific molecule at a weight-based dose of about 5 mg/kg once every 3 weeks for the duration of treatment. Another non-limiting example of a dosing regimen comprises administration of a PD-1×CTLA-4 bispecific molecule at a weight-based dose of about 6 mg/kg once every 3 weeks for the duration of treatment. Another non-limiting example of a dosing regimen comprises administration of a PD-1×CTLA-4 bispecific molecule at a weight-based dose of about 6.5 mg/kg once every 3 weeks for the duration of treatment. Another non-limiting example of a dosing regimen comprises administration of a PD-1×CTLA-4 bispecific molecule at a weight-based dose of about 7 mg/kg once every 3 weeks for the duration of treatment. Another non-limiting example of a dosing regimen comprises administration of a PD-1×CTLA-4 bispecific molecule at a weight-based dose of about 7.5 mg/kg once every 3 weeks for the duration of treatment. Another non-limiting example of a dosing regimen comprises administration of a PD-1×CTLA-4 bispecific molecule at a weight-based dose of about 8 mg/kg once every 3 weeks for the duration of treatment. Another non-limiting example of a dosing regimen comprises administration of a PD-1×CTLA-4 bispecific molecule at a weight-based dose of about 8.5 mg/kg once every 3 weeks for the duration of treatment. Another non-limiting example of a dosing regimen comprises administration of a PD-1×CTLA-4 bispecific molecule at a weight-based dose of about 9 mg/kg once every 3 weeks for the duration of treatment. Another non-limiting example of a dosing regimen comprises administration of a PD-1×CTLA-4 bispecific molecule at a weight-based dose of about 9.5 mg/kg once every 3 weeks for the duration of treatment. Another non-limiting example of a dosing regimen comprises administration of a PD-1×CTLA-4 bispecific molecule at a weight-based dose of about 10 mg/kg once every 3 weeks for the duration of treatment.

Generally, in the above embodiments, administration occurs at the predetermined frequency or periodicity, or within 1-3 days of such scheduled interval, such that administration occurs 1-3 days before, 1-3 days after, or on the day of a scheduled dose, e.g., once every 3 weeks (±3 days).

In the above embodiments, the PD-1×CTLA-4 bispecific molecule is administered by IV infusion. In such embodiments, the PD-1×CTLA-4 bispecific molecule is typically diluted into an infusion bag comprising a suitable diluent, e.g., saline. Since infusion or allergic reactions may occur, premedication for the prevention of such infusion reactions is recommended and precautions for anaphylaxis should be observed during the antibody administration. In certain embodiments, the IV infusion to be administered to the subject over a period of between about 30 minutes and about 4 hours. In certain embodiments, the IV infusion is delivered over a period of about 30-240 minutes, about 30-180 minutes, about 30-120 minutes, or about 30-90 minutes, or over a period of about 30-60 minutes, or over a period of about 45-60 minutes, or over a lesser period, if the subject does not exhibit signs or symptoms of an adverse infusion reaction. In a specific embodiment, the IV infusion is delivered over a period of about 45-60 minutes.

### V. Pharmaceutical Compositions

A PD-1×CTLA-4 bispecific molecule of the invention (e.g., DART-D) can be formulated in a composition. The compositions of the invention include bulk drug compositions useful in the manufacture of non-pharmaceutical compositions (e.g., impure or non-sterile compositions) and pharmaceutical compositions (i.e., compositions that are suitable for administration to a subject or patient) that can be used in the preparation of unit dosage forms. Such compositions comprise a prophylactically or therapeutically effective amount of a PD-1×CTLA-4 bispecific molecule of the present invention and one or more pharmaceutically acceptable carrier(s) and may optionally additionally include one or more additional therapeutic agents. The pharmaceutical compositions may be supplied, for example, as an aqueous solution, or a dry lyophilized powder or water-free concentrate specifically adapted for reconstitution with such a pharmaceutically acceptable carrier or reconstituted with such a carrier.

As used herein, the term “pharmaceutically acceptable carrier” means a diluent, solvent, dispersion media, antibacterial and antifungal agents, excipient, or vehicle approved by a regulatory agency of the Federal or a state government or listed in the U.S. Pharmacopeia or other generally recognized pharmacopeia as being suitable for administration to animals, and more particularly to humans. Such pharmaceutical carriers can be sterile liquids, such as water and oils, including those of petroleum, animal, vegetable or synthetic origin. Saline solutions and aqueous dextrose and glycerol solutions can also be employed as liquid carriers, particularly for injectable solutions. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions are supplied either separately or mixed together in a dose form, for example, as a dry lyophilized powder or water-free concentrate, or as an aqueous solution in a hermetically sealed container such as a bottle, vial, ampoule or sachet indicating the quantity of active agent. Where the composition is to be administered by infusion, it can be dispensed with an infusion bottle containing sterile pharmaceutical grade water or saline. Where the composition is administered by injection, an ampoule of sterile water for injection, saline or other diluent can be provided so that the ingredients may be mixed prior to administration.

### VII. Pharmaceutical Kits

The invention also provides a pharmaceutical pack or kit comprising one or more containers containing a pharmaceutical composition or pharmaceutical compositions and instructional material (e.g., a notice, package insert, instruction, etc.). Additionally, one or more other prophylactic or therapeutic agents useful for the treatment of a disease can also be included in the pharmaceutical kit. The containers of such pharmaceutical kits may, for example, comprise one or more hermetically sealed bottles, vials, ampoules, sachets, etc., indicating the quantity of active agent contained therein. Where the composition is to be administered by infusion, the container may be an infusion bottle, bag, etc. containing a sterile pharmaceutical-grade solution (e.g., water, saline, a buffer, etc.). Where the compositions are to be administered by injection, the pharmaceutical kit may contain an ampoule of sterile water, saline or other diluent for injection, so as to facilitate the mixing of the components of the pharmaceutical kit for administration to a subject (e.g., a human patient or other mammal). In certain embodiments, a pharmaceutical pack or kit comprises a PD-1×CTLA-4 bispecific molecule-containing pharmaceutical composition and instructional material.

In one embodiment, a PD-1×CTLA-4 bispecific molecule (e.g., DART-D) of such kit is/are supplied as a dry sterilized lyophilized powder or water-free concentrate in a hermetically sealed container and can be reconstituted, e.g., with water, saline, or other diluent to the appropriate concentration for administration to a subject. In certain embodiments, a PD-1×CTLA-4 bispecific molecule (e.g., DART-D) of such kit is supplied as an aqueous solution in a hermetically sealed container and can be diluted, e.g., with water, saline, or other diluent, to the appropriate concentration for administration to a subject. The kit can further comprise one or more other prophylactic and/or therapeutic agents useful for the treatment of cancer, in one or more containers; and/or the kit can further comprise one or more cytotoxic antibodies that bind one or more cancer antigens associated with cancer. In certain embodiments, the other prophylactic or therapeutic agent is a chemotherapeutic agent. In certain embodiments, the prophylactic or therapeutic agent is a biological agent or hormonal therapeutic agent.

A kit sometimes includes instructions and/or descriptions for carrying out a process described herein, which is referred to herein as “instructional material,” and in some embodiments, instructional material is provided in tangible form or electronic form. In certain embodiments, instructional material is provided as an electronic storage data file present on a suitable computer readable storage medium, e.g., portable flash drive, DVD, CD-ROM, diskette, and the like. In certain embodiments, a kit includes a written description of an internet location that provides instructional material in electronic form. The included instructional material of the pharmaceutical kits may, for example, be of a content and format prescribed by a governmental agency regulating the manufacture, use or sale of pharmaceuticals or biological products, and may indicate approval by the agency of the manufacture, sale or use of the pharmaceutical composition for human administration and/or for human therapy. The instructional material may, for example provide information relating to the contained dose of the pharmaceutical composition, modes of how it may be administered, etc. Such instructions may further provide information relating to the dose and administration of one or more pharmaceutical composition that are not provided in the kit.

Thus, for example, the included instructional material of the pharmaceutical kits may instruct that the provided pharmaceutical compositions are to be administered in combination with an additional agent which may be provided in the same pharmaceutical kit or in a separate pharmaceutical kit. Such instructional material may instruct that the provided PD-1×CTLA-4 bispecific molecule pharmaceutical composition comprises, or is to be reconstituted to administer a dose of about 3 mg/kg to about 10 mg/kg, about 3 mg/kg to about 8 mg/kg, about 3 mg/kg to about 6 mg/kg, about 6 mg/kg to about 10 mg/kg, about 6 mg/kg to about 9 mg/kg, about 6 mg/kg to about 8 mg/kg, about 6 mg/kg to about 7 mg/kg, about 7 mg/kg to about 8 mg/kg, about 8 mg/kg to about 9 mg/kg, or about 9 mg/kg to about 10 mg/kg. Such instructional material may instruct that the provided PD-1×CTLA-4 bispecific molecule pharmaceutical composition comprises, or is to be reconstituted to administer a dose of about 3 mg/kg, about 4 mg/kg, about 5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. Such instructional material may instruct that the provided PD-1×CTLA-4 bispecific molecule pharmaceutical composition is to be administered, once about every 3 weeks, about once every 6 weeks, or a combination thereof. Such instructional material may instruct that the provided PD-1×CTLA-4 bispecific molecule pharmaceutical composition is to be administered at a specified dose and interval during an induction period. Such instructional material may further instruct that the provided PD-1×CTLA-4 bispecific molecule pharmaceutical composition is to be administered at a specified dose and interval during a subsequent maintenance period. In certain embodiments, such instructional material instructs that the dose administered during a maintenance period is the same as the dose administered during an induction period. In certain embodiments, such instructional material instructs that the dose administered during a maintenance period is different from the dose administered during an induction period. In certain embodiments, such instructional material instructs that the dosing interval during a maintenance period is different from the dosing interval during an induction period. In certain embodiments, such instructional material instructs that the dosing interval during a maintenance period is the same as the dosing interval during an induction period. In certain embodiments, such instructional material instructs that the dose administered and the dosing interval are unchanged during the course of treatment. Such instructional material may instruct regarding the mode of administration of the included pharmaceutical composition, for example that it is to be administered by intravenous (IV) infusion. The included instructional material of the pharmaceutical kits may instruct regarding the duration or timing of such administration, for example that the included pharmaceutical composition is composition is to be administered by intravenous (IV) infusion over a period of about 30 minutes, about 45 minutes, about 60 minutes, about 30-240 minutes, a period of about 30-90 minutes, etc.

The included instructional material of the pharmaceutical kits may instruct regarding the appropriate or desired use of the included pharmaceutical composition, for example instructing that such pharmaceutical composition is to be administered for the treatment of cancer. Such cancer may be an adrenal gland cancer, an AIDS-associated cancer, an alveolar soft part sarcoma, an astrocytic tumor, an anal cancer a bile duct cancer, a bladder cancer, a bone cancer, a brain cancer, a brain and spinal cord cancer, a breast cancer, a HER2+ breast cancer, a triple negative breast cancer (TNBC), a carotid body tumors, a cervical cancer, an HPV-related cervical cancer, a cervical squamous cell carcinoma, a chondrosarcoma, a chordoma, a clear cell carcinoma, a colon cancer, a colorectal cancer (CRC), a microsatellite instability-high colorectal cancer (MSI-H CRC), a microsatellite-stable colorectal cancer (non-microsatellite-instability-high colorectal cancer, non-MSI-H CRC), a desmoplastic small round cell tumor, an ependymoma, a Ewing's tumor, an extraskeletal myxoid chondrosarcoma, a fallopian tube carcinoma, a fibrogenesis imperfecta ossium, a fibrous dysplasia of the bone, a gallbladder or bile duct cancer, a gastric cancer, a gestational trophoblastic disease, a germ cell tumor, a glioblastoma, a head and neck cancer, an HPV-related head and neck cancer, a hematological malignancy, a hepatocellular carcinoma, an islet cell tumor, a Kaposi's Sarcoma, a kidney cancer, a leukemia, an acute myeloid leukemia, a liposarcoma/malignant lipomatous tumor, a liver cancer, a lymphoma, a lung cancer, NSCLC, a medulloblastoma, a melanoma, a meningioma, Merkel cell carcinoma, a mesothelioma pharyngeal cancer, a multiple endocrine neoplasia, a multiple myeloma, a myelodysplastic syndrome, a neuroblastoma, a neuroendocrine tumors, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer, mCRPC, a posterior uveal melanoma, a renal cancer, a renal cell carcinoma (RCC), a rhabdoid tumor, a rhabdomyosarcoma, a sarcoma, a skin cancer, a small round blue cell tumor of childhood (including neuroblastoma and rhabdomyosarcoma), a soft-tissue sarcoma, a pleomorphic undifferentiated sarcoma, a dedifferentiated liposarcoma, a synovial sarcoma, a myxofibrosarcoma, a squamous cell cancer, SCCHN, a stomach cancer, a synovial sarcoma, a testicular cancer, a thymic carcinoma, a thymoma, a thyroid cancer, a thyroid metastatic cancer, and a uterine cancer.

### VII. Embodiments of the Invention

The invention concerns in part the following non-limiting embodiments (E1-E92):
E1. A method of treating a cancer comprising administering a PD-1×CTLA-4 bispecific molecule to a subject in need thereof, wherein said PD-1×CTLA-4 bispecific molecule comprises a PD-1 Binding Domain and a CTLA-4 Binding Domain, and wherein said method comprises administering said PD-1×CTLA-4 bispecific molecule to a subject at a dose of about 3 mg/kg to about 10 mg/kg once every 3 weeks.
E2. A method of stimulating immune cells comprising administering a PD-1×CTLA-4 bispecific molecule to a subject in need thereof, wherein said PD-1×CTLA-4 bispecific molecule comprises a PD-1 Binding Domain and a CTLA-4 Binding Domain, and wherein said method comprises administering said PD-1×CTLA-4 bispecific molecule to a subject at a dose of about 3 mg/kg to about 10 mg/kg once every 3 weeks.
E3. The method of E1 or E2, wherein said PD-1×CTLA-4 bispecific molecule is administered to said subject at a dose of about 3 mg/kg to about 10 mg/kg once every 3 weeks during an induction period.
E4. The method of E2 or E3, wherein said immune cells are T cells.
E5. The method of any one of E1-E4, wherein:
   (I) said PD-1 Binding Domain comprises a Light Chain Variable Domain (VL_{PD-1}) that comprises the CDR_{L}1, CDR_{L}2 and CDR_{L}3 of SEQ ID NO:1, and a Heavy Chain Variable Domain (VH_{PD-1}) that comprises the PD-1-specific CDR_{H}1, CDR_{H}2 and CDR_{H}3 of SEQ ID NO:5; and
   (II) said CTLA-4 Binding Domain comprises a Light Chain Variable Domain (VL_{CTLA-4}) that comprises the CDR_{L}1, CDR_{L}2 and CDR_{L}3 of SEQ ID NO:9, and a Heavy Chain Variable Domain (VH_{CTLA-4}) that comprises the CTLA-4-specific CDR_{H}1, CDR_{H}2 and CDR_{H}3 of SEQ ID NO:13.
E6. The method of any one of E1-E5, wherein said PD-1×CTLA-4 bispecific molecule comprises:
   (I) two of said PD-1 Binding Domains; and
   (II) two of said CTLA-4 Binding Domains.
E7. The method of any one of E1-E6, wherein said PD-1 Binding Domain comprises the VL Domain of SEQ ID NO:1 and the VH Domain of SEQ ID NO:5.
E8. The method of any one of E1-E7, wherein said CTLA-4 Binding Domain comprises the VL Domain of SEQ ID NO:9 and the VH Domain of SEQ ID NO:13.
E9. The method of any one of E1-E8, wherein said PD-1×CTLA-4 bispecific molecule comprises an Fc Region.
E10. The method of E9, wherein said Fc Region is of an IgG1, IgG2, IgG3, or IgG4 isotype.
E11. The method of any one of E9 or E10, wherein said PD-1×CTLA-4 bispecific molecule further comprises a Hinge Domain.
E12. The method of E11, wherein said Fc Region and said Hinge Doman are of the IgG4 isotype, and wherein said Hinge Domain comprises a stabilizing mutation.
E13. The method of any one of E9-E12, wherein said Fc Region is a variant Fc Region that comprises:
   (a) one or more amino acid modifications that reduces the affinity of the variant Fc Region for an FcγR; and/or
   (b) one or more amino acid modifications that enhances the serum half-life of the variant Fc Region.
E14. The method of E13, wherein said one or more amino acid modifications that reduces the affinity of the variant Fc Region for an FcγR comprise the substitution of L234A or L235A, or L234A and L235A, wherein said numbering is that of the EU index as in Kabat.
E15. The method of any one of E13 or E14, wherein said one or more amino acid modifications that enhances the serum half-life of the variant Fc Region comprise the substitution of M252Y; or M252Y and S254T; or M252Y and T256E; or M252Y, S254T and T256E; or K288D and H435K, wherein said numbering is that of the EU index as in Kabat.
E16. The method of any one of E1-E15, wherein said PD-1×CTLA-4 bispecific molecule is a diabody comprising one polypeptide chain that comprises the amino acid sequence of SEQ ID NO:40 and a second polypeptide chain that comprises the amino acid sequence of SEQ ID NO:41.
E17. The method of any one of E1-E64, wherein said PD-1×CTLA-4 bispecific molecule is a diabody comprising two polypeptide chains each comprising the amino acid sequence of SEQ ID NO:40 and two polypeptide chains each comprising the amino acid sequence of SEQ ID NO:41.
E18. The method of any one of E1-E18, wherein said PD-1×CTLA-4 bispecific molecule is administered at a dose of between about 3 mg/kg and 8 mg/kg.
E19. The method of any one of E1-E18, wherein said PD-1×CTLA-4 bispecific molecule is administered at a dose of between about 6 mg/kg and about 10 mg/kg.
E20. The method of any one of E1-E18, wherein said PD-1×CTLA-4 bispecific molecule is administered at a dose of about 3 mg/kg.
E21. The method of any one of E1-E18, wherein said PD-1×CTLA-4 bispecific molecule is administered at a dose of about 4 mg/kg.
E22. The method of any one of E1-E18, wherein said PD-1×CTLA-4 bispecific molecule is administered at a dose of about 5 mg/kg.
E23. The method of any one of E1-E19, wherein said PD-1×CTLA-4 bispecific molecule is administered at a dose of about 6 mg/kg.
E24. The method of any one of E1-E19, wherein said PD-1×CTLA-4 bispecific molecule is administered at a dose of about 6.5 mg/kg.
E25. The method of any one of E1-E19, wherein said PD-1×CTLA-4 bispecific molecule is administered at a dose of about 7 mg/kg.
E26. The method of any one of E1-E19, wherein said PD-1×CTLA-4 bispecific molecule is administered at a dose of about 7.5 mg/kg.
E27. The method of any one of E1-E19, wherein said PD-1×CTLA-4 bispecific molecule is administered at a dose of about 8 mg/kg.
E28. The method of any one of E1-E17 or E19, wherein said PD-1×CTLA-4 bispecific molecule is administered at a dose of about 8.5 mg/kg.
E29. The method of any one of E1-E17 or E19, wherein said PD-1×CTLA-4 bispecific molecule is administered at a dose of about 9 mg/kg.
E30. The method of any one of E1-E17, or E19, wherein said PD-1×CTLA-4 bispecific molecule is administered at a dose of about 9.5 mg/kg.
E31. The method of any one of E3-E16, further comprising administering said PD-1×CTLA-4 bispecific molecule to said subject at a dose of from about 3 mg/kg to about 10 mg/kg once every 6 weeks during a maintenance period, wherein said maintenance period follows said induction period.
E32. The method of any one of E3-E17 or E31, wherein said induction period has a duration of up to about 24 weeks.
E33. The method of any one of E3-E17, or E31-E32, wherein said maintenance period has a duration of up to about 84 weeks.
E34. The method of any one of E3-E17, or E31-E33, wherein said PD-1×CTLA-4 bispecific molecule is administered at a dose of between about 3 mg/kg and 8 mg/kg during said induction period.
E35. The method of any one of E3-E17, or E31-E33, wherein said PD-1×CTLA-4 bispecific molecule is administered at a dose of between about 6 mg/kg and about 10 mg/kg during said induction period.
E36. The method of any one of E3-E17, or E31-E34, wherein said PD-1×CTLA-4 bispecific molecule is administered at a dose of about 3 mg/kg during said induction period.
E37. The method of any one of E3-E17, or E31-E34, wherein said PD-1×CTLA-4 bispecific molecule is administered at a dose of about 4 mg/kg during said induction period.
E38. The method of any one of E3-E17, or E31-E34, wherein said PD-1×CTLA-4 bispecific molecule is administered at a dose of about 5 mg/kg during said induction period.
E39. The method of any one of E3-E17, or E31-E35, wherein said PD-1×CTLA-4 bispecific molecule is administered at a dose of about 6 mg/kg during said induction period.
E40. The method of any one of E3-E17, or E31-E35, wherein said PD-1×CTLA-4 bispecific molecule is administered at a dose of about 6.5 mg/kg during said induction period.
E41. The method of any one of E3-E17, or E31-E35, wherein said PD-1×CTLA-4 bispecific molecule is administered at a dose of about 7 mg/kg during said induction period.
E42. The method of any one of E3-E17, or E31-E35, wherein said PD-1×CTLA-4 bispecific molecule is administered at a dose of about 7.5 mg/kg during said induction period.
E43. The method of any one of E3-E17, or E31-E35, wherein said PD-1×CTLA-4 bispecific molecule is administered at a dose of about 8 mg/kg during said induction period.
E44. The method of any one of E3-E17, E31-E33, or E35, wherein said PD-1×CTLA-4 bispecific molecule is administered at a dose of about 8.5 mg/kg during said induction period.
E45. The method of any one of E3-E17, E31-E33, or E35, wherein said PD-1×CTLA-4 bispecific molecule is administered at a dose of about 9 mg/kg during said induction period.
E46. The method of any one of E3-E17, E31-E33, or E35, wherein said PD-1×CTLA-4 bispecific molecule is administered at a dose of about 9.5 mg/kg during said induction period.
E47. The method of any one of E3-E17, E31-E33, or E35, wherein said PD-1×CTLA-4 bispecific molecule is administered at a dose of about 10 mg/kg during said induction period.
E48. The method of any one of E31-E47, wherein said PD-1×CTLA-4 bispecific molecule is administered at a dose of between about 3 mg/kg and 8 mg/kg during said maintenance period.
E49. The method of any one of E31-E47, wherein said PD-1×CTLA-4 bispecific molecule is administered at a dose of between about 6 mg/kg and about 10 mg/kg during said maintenance period.
E50. The method of any one of E31-E48, wherein said PD-1×CTLA-4 bispecific molecule is administered at a dose of about 3 mg/kg during said maintenance period.
E51. The method of any one of E31-E48, wherein said PD-1×CTLA-4 bispecific molecule is administered at a dose of about 4 mg/kg during said maintenance period.
E52. The method of any one of E31-E48, wherein said PD-1×CTLA-4 bispecific molecule is administered at a dose of about 5 mg/kg during said maintenance period.
E53. The method of any one of E31-E49, wherein said PD-1×CTLA-4 bispecific molecule is administered at a dose of about 6 mg/kg during said maintenance period.
E54. The method of any one of E31-E49, wherein said PD-1×CTLA-4 bispecific molecule is administered at a dose of about 6.5 mg/kg during said maintenance period.
E55. The method of any one of E31-E49, wherein said PD-1×CTLA-4 bispecific molecule is administered at a dose of about 7 mg/kg during said maintenance period.
E56. The method of any one of E31-E49, wherein said PD-1×CTLA-4 bispecific molecule is administered at a dose of about 7.5 mg/kg during said maintenance period.
E57. The method of any one of E31-E49, wherein said PD-1×CTLA-4 bispecific molecule is administered at a dose of about 8 mg/kg during said maintenance period.
E58. The method of any one of E31-E47, or E49, wherein said PD-1×CTLA-4 bispecific molecule is administered at a dose of about 8.5 mg/kg during said maintenance period.
E59. The method of any one of E31-E47, or E49, wherein said PD-1×CTLA-4 bispecific molecule is administered at a dose of about 9 mg/kg during said maintenance period.
E60. The method of any one of E31-E47, or E49, wherein said PD-1×CTLA-4 bispecific molecule is administered at a dose of about 9.5 mg/kg during said maintenance period.
E61. The method of any one of E31-E47, or E49, wherein said PD-1×CTLA-4 bispecific molecule is administered at a dose of about 10 mg/kg during said maintenance period.
E62. The method of any one of E31-E61, wherein said dose of said PD-1×CTLA-4 bispecific molecule administered in said maintenance period is the same as said dose administered in said induction period.
E63. The method of any one of E31-E61, wherein said dose of said PD-1×CTLA-4 bispecific molecule administered in said maintenance period is different than said dose administered in said induction period.
E64. The method of any one of E1-E63, wherein said PD-1×CTLA-4 bispecific molecule is administered by intravenous (IV) infusion.
E65. The method of E64, wherein said IV infusion is over a period of between about 30 minutes to about 60 minutes.
E66. The method of any one of E1-E65, wherein said cancer is selected from the group consisting of: an adrenal gland cancer, an AIDS-associated cancer, an alveolar soft part sarcoma, an astrocytic tumor, an anal cancer, a bile duct cancer, a bladder cancer, a bone cancer, a brain cancer, a brain and spinal cord cancer, a breast cancer, a HER2+ breast cancer, a triple negative breast cancer (TNBC), a carotid body tumors, a cervical cancer, an HPV-related cervical cancer, a cervical squamous cell carcinoma, a chondrosarcoma, a chordoma, a clear cell carcinoma, a colon cancer, a colorectal cancer (CRC), a microsatellite instability-high colorectal cancer (MSI-H CRC), a microsatellite-stable colorectal cancer (non-microsatellite-instability-high colorectal cancer, non-MSI-H CRC), a desmoplastic small round cell tumor, an endometrial cancer, an ependymoma, a Ewing's tumor, an extraskeletal myxoid chondrosarcoma, a fallopian tube carcinoma, a fibrogenesis imperfecta ossium, a fibrous dysplasia of the bone, a gallbladder or bile duct cancer, a gastric cancer, a gestational trophoblastic disease, a germ cell tumor, a glioblastoma, a head and neck cancer, an HPV-related head and neck cancer, a hematological malignancy, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a non-small-cell lung cancer (NSCLC), a medulloblastoma, a melanoma, a meningioma, Merkel cell carcinoma, a mesothelioma pharyngeal cancer, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer, a metastatic castration resistant prostate cancer (mCRPC), a posterior uveal melanoma, a renal cancer, a renal cell carcinoma (RCC), a rhabdoid tumor, a rhabdomyosarcoma, a sarcoma, a skin cancer, a small round blue cell tumor of childhood (including neuroblastoma and rhabdomyosarcoma), a soft-tissue sarcoma, a pleomorphic undifferentiated sarcoma, a dedifferentiated liposarcoma, a synovial sarcoma, a myxofibrosarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.
E67. The method of E66, wherein said cancer is selected from the group consisting of: cervical cancer, HPV-related cervical cancer, cervical squamous cell carcinoma, CRC, MSI-H CRC, non-MSI-H CRC, head and neck cancer, HPV-related head and neck cancer, lung cancer, melanoma, NSCLC, prostate cancer, renal cancer, RCC, soft-tissue sarcoma, a pleomorphic undifferentiated sarcoma, a dedifferentiated liposarcoma, a synovial sarcoma, a myxofibrosarcoma, squamous cell cancer, and SCCHN.
E68. The method of any one of E66 or E67, wherein said cancer is cervical cancer.
E69. The method of any one of E66 or E67, wherein said cancer is cervical squamous cell carcinoma.
E70. The method of any one of E66 or E67, wherein said cancer is CRC.
E71. The method of any one of E66-E67 or E70, wherein said CRC is non-MSI-H CRC.
E72. The method of any one of E66-E67 or E70, wherein said CRC is a MSI-H CRC.
E73. The method of any one of E66 or E67, wherein said cancer is lung cancer.
E74. The method of any one of E66-E67 or E73, wherein said lung cancer is NSCLC.
E75. The method of any one of E66 or E67, wherein said cancer is melanoma.
E76. The method of any one of E66-E67 or E75, wherein said melanoma is cutaneous melanoma.
E77. The method of any one of E66 or E67, wherein said cancer is prostate cancer.
E78. The method of any one of E66-E67 or E77, wherein the prostate cancer is metastatic castration-resistant prostate cancer (mCRPC).
E79. The method of any one of E66 or E67, wherein said cancer is renal cancer.
E80. The method of any one of E66-E67 or E79, wherein said renal cancer is RCC.
E81. The method of any one of E66 or E67, wherein said cancer is soft tissue sarcoma.
E82. The method of any one of E66-E67 or E81, wherein said cancer is pleomorphic undifferentiated sarcoma, dedifferentiated liposarcoma, synovial sarcoma, or myxofibrosarcoma.
E83. The method of any one of E66 or E67, wherein said cancer is squamous cell cancer.
E84. The method of any one of E66 or E67, wherein said cancer is head and neck cancer.
E85. The method of any one of E66-E67 or E84, wherein said squamous cell cancer or said head and neck cancer is SCCHN.
E86. The method of any one of E1-E85, further comprising administering a therapeutically or prophylactically effective amount of one or more additional therapeutic agents or chemotherapeutic agents.
E87. The method of any one of E1-E86, wherein said subject in need thereof is a human.
E88. A pharmaceutical kit comprising:
   (a) a container comprising a PD-1×CTLA-4 bispecific molecule; and
   (b) an instructional material, wherein the instructional material instructs that said PD-1×CTLA-4 bispecific molecule is to be used according to the method of any one of E1-E87.
E89. Use of the pharmaceutical kit of E88 for the treatment of cancer.
E90. Use of the pharmaceutical kit of E88 for stimulating immune cells.
E91. Use of a PD-1×CTLA-4 bispecific molecule according to the method of any one of E1 or E3-E87 for the treatment of cancer.
E92. Use of a PD-1×CTLA-4 bispecific molecule according to the method of any one of E2-E87 for stimulating immune cells.

### EXAMPLES

Having now generally described the invention, the same will be more readily understood through reference to the following Examples. The following examples illustrate various methods for compositions in the diagnostic or treatment methods of the invention. The examples are intended to illustrate, but in no way limit, the scope of the invention.

### Example 1

### The PD-1×CTLA-4 Bispecific Molecule DART-D Provides Optimal Dual PD-1 and CTLA-4 Checkpoint Blockade In Vitro

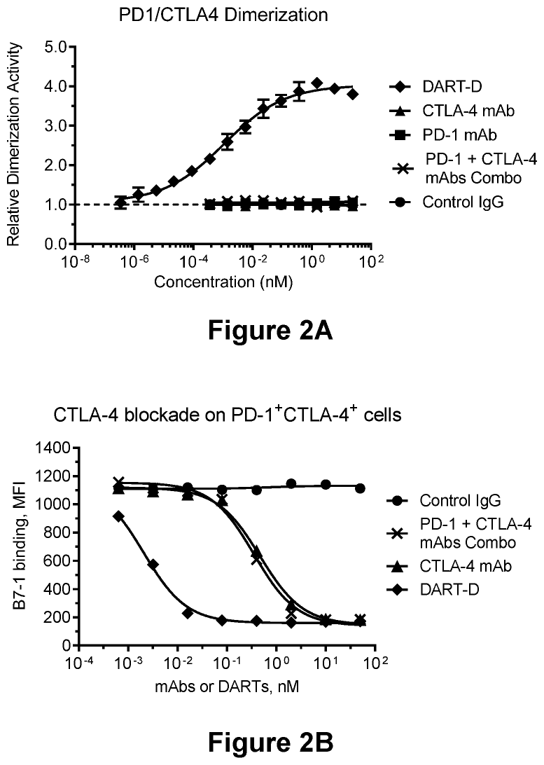

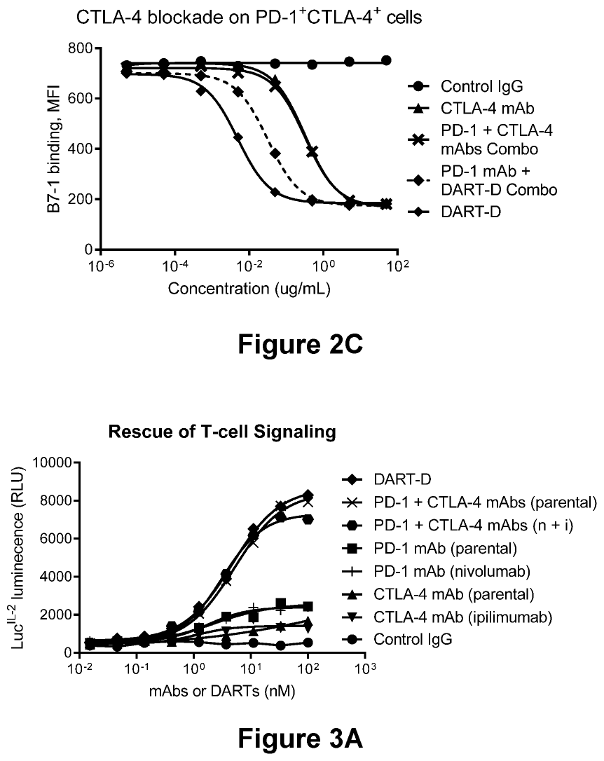

Using the DART platform (Huang, L, et al. (2020) “Multispecific, Multivalent Antibody-Based Molecules Engineered on the DART® and TRIDENT™ Platforms.” Curr Protoc Immunol. 2020; 129(1):e95), a tetravalent (2×2 format) PD-1×CTLA-4 bispecific molecule was created from domains of two high affinity, ligand-blocking monoclonal antibodies (mAbs) and an IgG4 backbone to limit Fc-dependent effector functions, the general structure is shown in FIG. 1 and the amino acid sequence of each polypeptide chain is provided above (see, e.g., Table 1). DART-D is able to interact in cis with both PD-1 and CTLA-4 receptors on the same cell. As shown in FIG. 2A, enzyme complementation (using the PathHunter® PD-1⁺CTLA-4⁺ assay) was observed following DART-D mediated co-ligation of PD-1 and CTLA-4 expressed on the surface of model cells demonstrates that a single molecule of DART-D is capable of simultaneous engagement of PD-1 and CTLA-4 on a single cell. In contrast no enzyme complementation was observed using a combination of PD-1 and CTLA-4 mAbs. As shown in FIG. 2B, the avidity contributed by cis-mode binding to the two antigens results in greatly enhanced DART-D-mediated blockade of CTLA-4 activity on dual-expressing cells, with IC₅₀ improved ˜100 fold over its parental mAb. In the presence of 10-fold excess of parental anti-PD-1 mAb DART-D demonstrated approximately 10-fold reduction in CTLA-4 blocking activity (FIG. 2C), indicating that the enhanced CTLA-4 blockade on dual-expressing cells is due to an avidity effect mediated by “anchoring” of DART-D via its PD-1 arm. These studies demonstrate that DART-D is able to engage PD-1 and CTLA-4 independently as well as co-engage both checkpoints on a surface of cells that coexpress them leading to a differential degree of CTLA-4 blockade.

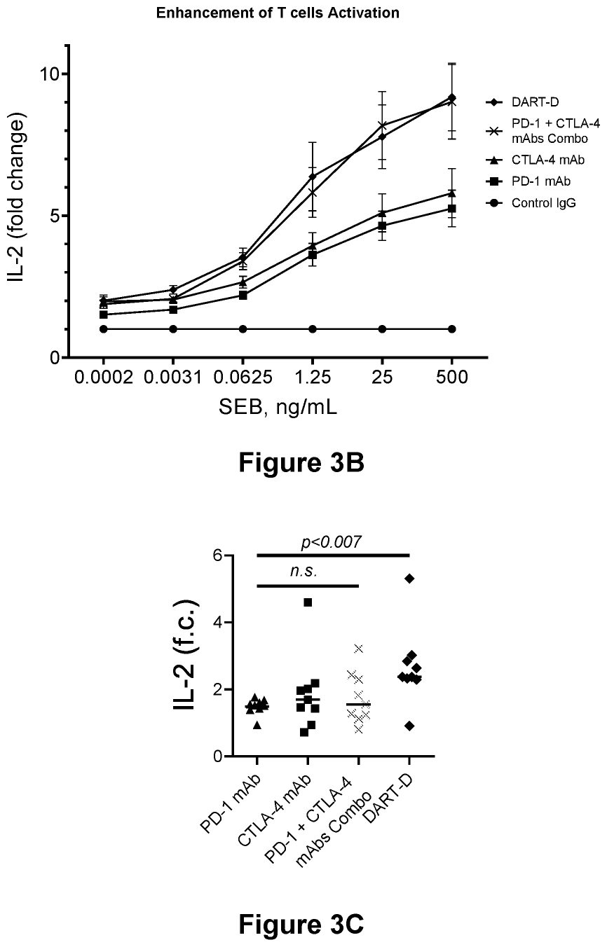

To determine the ability to overcome dual PD-1/CTLA-4 checkpoint suppression in T cells, DART-D was evaluated-side-by-side with PD-1 and CTLA-4 mAb combinations in an engineered reporter assay (FIG. 3A) and in primary SEB T-cell activation assays (FIG. 3B). In both assay systems, DART-D supported dual checkpoint pathway reversal to the same level as mAb combinations, including replicas of ipilimumab and nivolumab. In approximately a quarter of healthy donors, where PD-1 or CTLA-4 blockade with individual blocking mAbs did not substantially impact SEB-driven T-cell activation, but DART-D, but not the combination of two mAbs, consistently enhanced the TL-2 release (FIG. 3C).

### Example 2

### Evaluation of the PD-1×CTLA-4 Bispecific Molecule DART-D in Non-Human Primates

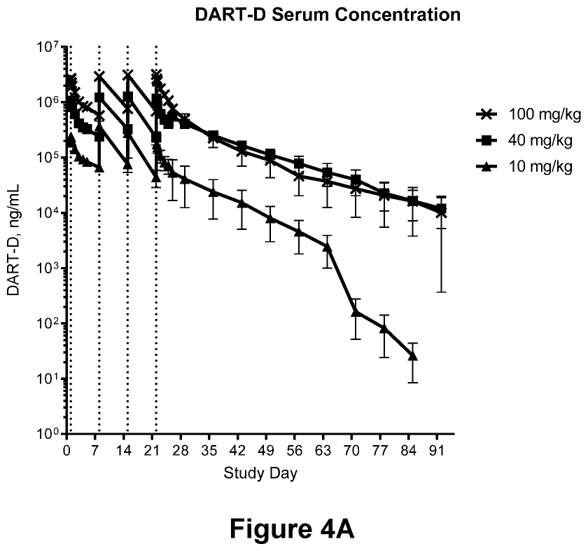

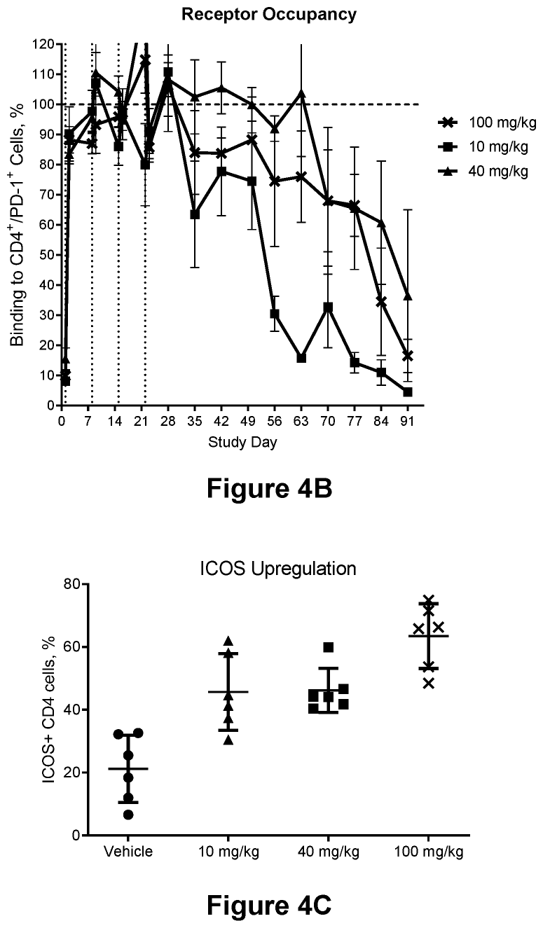

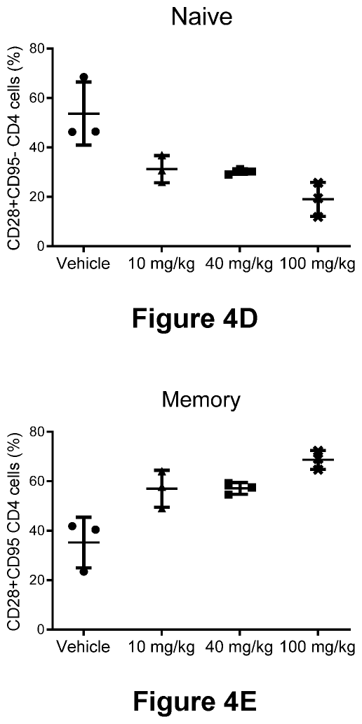

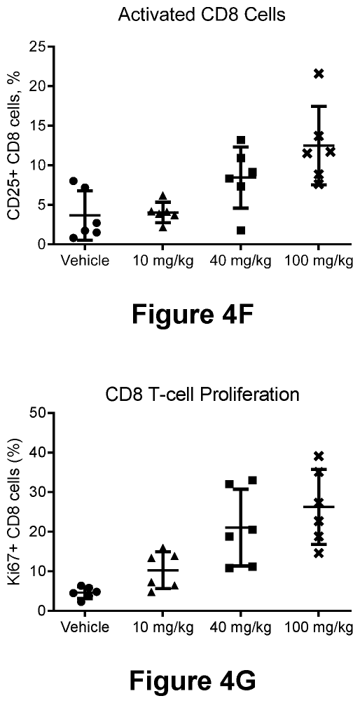

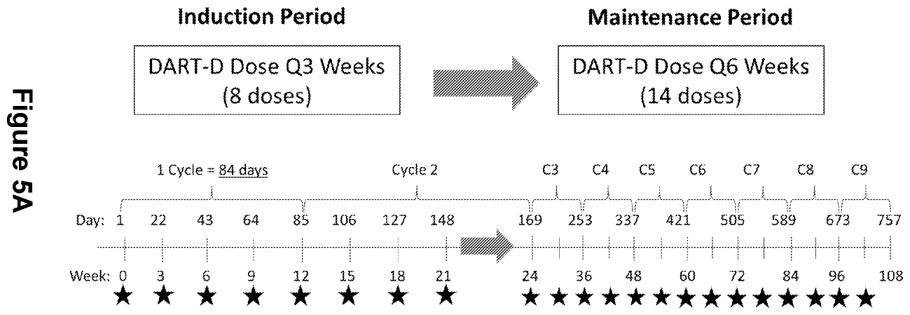

To determine pharmacokinetic (PK)/pharmacodynamic (PD) and toxicity profile, DART-D was evaluated in cynomolgus monkey, a relevant cross-reactive species. DART-D demonstrated linear PK (half-life ˜7 days) across the tested dose range of 10-100 mg/kg (FIG. 4A). All animals from the individual dose groups achieved comparable exposure to DART-D during the first dose interval; however, exposure decreased in some animals during the fourth dose due to the appearance of anti-drug antibodies (ADA). Repeated intravenous administrations (4 weekly doses) of DART-D were well-tolerated at dose levels of 10, 40 and 100 mg/kg. In-life effects were limited to increased incidence of soft/watery feces at ≥40 mg/kg/dose and minor hematological changes. There were no DART-D-related effects on body weights, food consumption, veterinary physical examinations, or gross necropsy observations. Spleen weight parameters were increased for males at doses of ≥40 mg/kg and females at dose of ≥10 mg/kg, which correlated microscopically with generalized lymphoid hyperplasia. All effects were reversible following a 10-week recovery period and were not considered adverse. The no-observed-adverse-effect level was declared at 100 mg/kg, the highest dose tested. By contrast, previous studies reported the combination of ipilimumab+nivolumab, resulted in diarrhea and GI tract inflammation at doses as low as 3 mg/kg (ipilimumab) and 10 mg/kg (nivolumab), and the 10 (ipilimumab) and 50 mg/kg (nivolumab) dose exceeded the highest non-severely toxic dose (Selby, M. J., et al., 2016. “Preclinical Development of Ipilimumab and Nivolumab Combination Immunotherapy: Mouse Tumor Models, In Vitro Functional Studies, and Cynomolgus Macaque Toxicology.” PLoS One, 11: e0161779).

The level of DART-D binding to PD-1-expressing circulating T cells correlated with serum concentration (FIG. 4B). A dose-dependent increase in fraction of splenic CD4⁺ T cells expressing ICOS was observed (FIG. 4C). In addition, a shift of in the relative proportion of circulating T-cells with a naïve phenotype (FIG. 4D) to memory-like phenotype (FIG. 4E) was observed in the animals, and there were no changes in tissue-resident or circulating T_{reg} population. These PD changes are consistent with previously reported effects of CTLA-4 blockade in vivo (Ng Tang, D., et al., 2013. “Increased frequency of ICOS+ CD4 T cells as a pharmacodynamic biomarker for anti-CTLA-4 therapy.” Cancer Immunol Res, 1: 229-34; Hokey, D. A., et al., 2008. “CLTA-4 blockade in vivo promotes the generation of short-lived effector CD8 T cells and a more persistent central memory CD4 T cell response.” J Med Primatol, 37 Suppl 2: 62-8). DART-D treatment was also associated with enhanced T-cell activation (FIG. 4F) and proliferation (FIG. 4G), indicating the impact of CTLA-4 blockade by a PD-1×CTLA-4 bispecific molecule. In sum, the PD changes are consistent with dual PD-1 and CTLA-4 blockade, and no excess toxicity was observed in cynomolgus monkeys.

### Example 3

### Phase I Dose Studies

In order to determine the tolerability of patients to the PD-1×CTLA-4 Bispecific Molecule, DART-D, a Phase I clinical study is conducted. The study includes a Dose Escalation phase and a Cohort Expansion phase. The study is approved by the institutional review boards of each clinical site, and all patients sign a written-informed consent. The clinical study was approved by IntegReview IRB, and registered on www.clinicaltrials.gov (Identifier: NCT03761017).

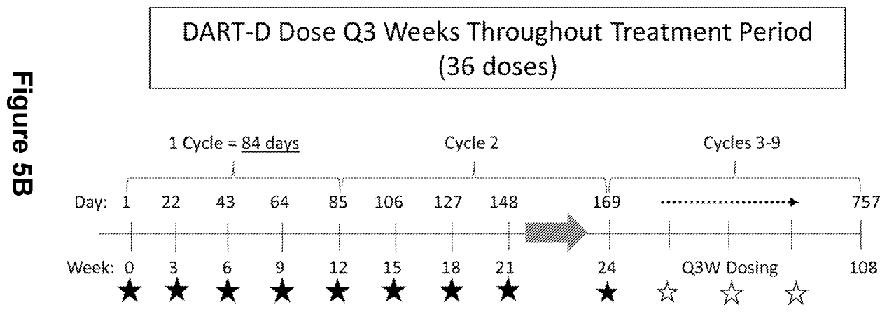

For the initial Dose Escalation and Dose Expansion cohorts, DART-D is administered once every three weeks (Q3W) during a 24-week Induction Period. For purposes of the study, a 12 week (84-day±3 days) cycle is used. After 24 weeks, or 2 cycles of therapy, clinically stable patients without toxicity that necessitates discontinuation or confirmed progressive disease (PD) proceed to a Maintenance Period. During the Maintenance Period, DART-D is administered every 6 weeks (Q6W). Patients receive up to 14 additional DART-D infusions (seven (7) additional 12-week Q6W treatment cycles) depending on tolerability and response to study treatments for a total of up to 9, 84-day cycles (i.e., total of 22 infusions). DART-D is administered by IV infusion over 30 minutes (up to 45 minutes) The treatment schema is presented in FIG. 5.

Target and non-target lesions are designated at screening and then evaluated at 12 and 18 weeks after treatment initiation during the Induction Period. During the Maintenance Period, tumor assessments occur every 12 weeks (±7 days). Following the last dose of study drug, all patients are followed for survival and tumor assessments. Tumor assessments are obtained using CT and/or MRI scans (cutaneous lesions may be measured using calipers and/or photographs with an included scale). Antitumor activity is evaluated according to conventional Response Evaluation Criteria in Solid Tumors (RECIST), version 1.1 (Eisenhauer, E. A., et al. (2009) “New Response Evaluation Criteria In Solid Tumours: Revised RECIST Guideline (Version 1.1),” Eur. J. Cancer. 45(2):228-247) e.

In the Dose Escalation phase, sequential escalating doses from 0.03 mg/kg up to 10 mg/kg are administered Q3W (Induction Period) following a conventional 3+3+3 design: successive cohorts of 3 to 9 patients each are evaluated (Table 2). Following the 24 week Induction Period DART-D is administered every 6 weeks during the Maintenance Period (FIG. 5). At various dose levels, patients assessed to not be evaluable for Dose Escalation purposes are replaced. Additional patients are enrolled at multiple dose levels of interest to gain additional clinical experience. In the Dose Escalation phase, patients with solid tumors of any histology are enrolled.

TABLE 2
Dose Escalation Cohorts

| Cohort | DART-D Dose (Q3W) |
|---|---|
| Cohort 1 | 0.03 mg/kg |
| Cohort 2 | 0.1 mg/kg |
| Cohort 3 | 0.3 mg/kg |
| Cohort 4 | 1.0 mg/kg |
| Cohort 5 | 3.0 mg/kg |
| Cohort 6 | 6.0 mg/kg |
| Cohort 7 | 10.0 mg/kg |

Intermediate dose levels between about 3 mg/kg to about 10 mg/kg and alternative schedules may be explored independently or concurrently. In particular, exploration of doses between about 6 mg/kg and about 10 mg/kg is specifically contemplated.

In the Cohort Expansion phase, patients with non-small cell lung cancer (NSCLC), squamous cell carcinoma of the head and neck (SCCHN), renal cell carcinoma (RCC), cervical cancer (particularly cervical squamous cell carcinoma), soft tissue sarcoma (particularly, pleomorphic undifferentiated sarcoma, dedifferentiated liposarcoma, synovial sarcoma and myxofibrosarcoma), and colorectal cancer (CRC) (particularly non-MSI-H CRC) receive DART-D at a dose selected based on the safety, PK and antitumor activity from the dose escalation phase of the study.

### Summary of Initial Findings

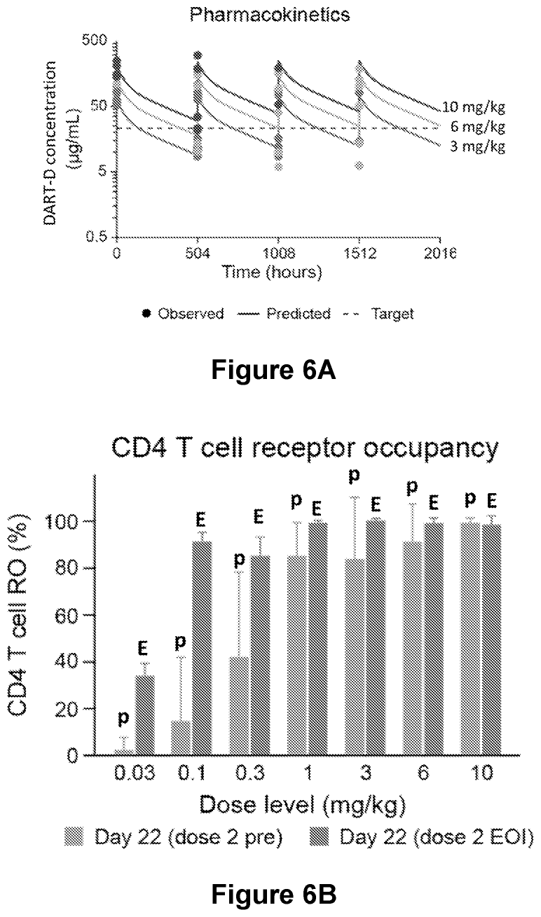

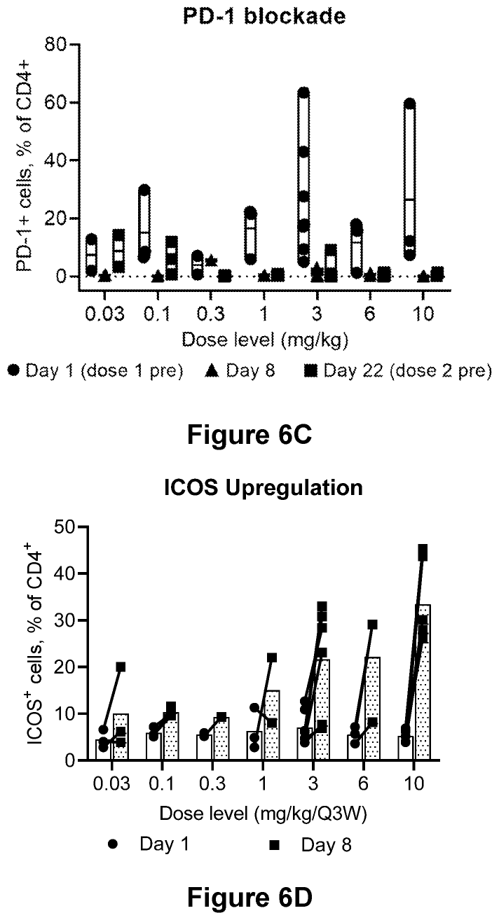

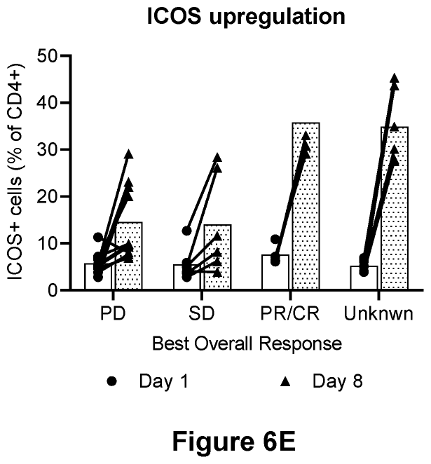

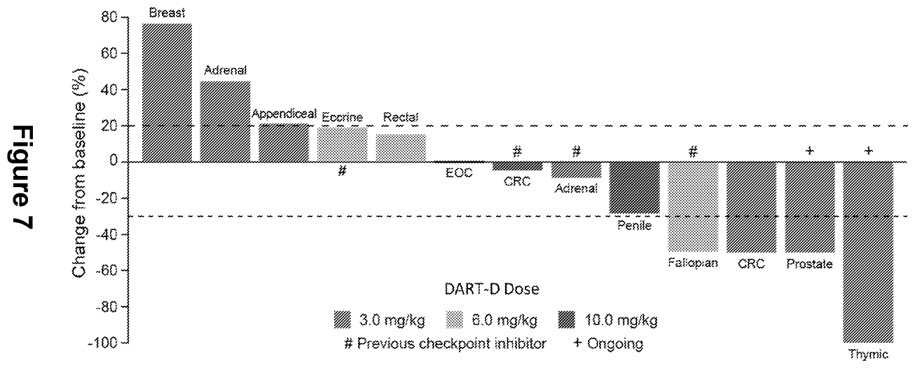

DART-D demonstrated linear kinetics with half-life equal to 12.4 days. Simulated multiple-dose PK profiles indicate that doses at or above 3 mg/kg maintain target serum trough concentrations of DART-D comparable to that of ipilimumab and nivolumab (see dashed line in FIG. 6A).

DART-D bound to circulating T cells (FIG. 6B) occupying PD-1 for durations proportional to dose and serum concentration (FIG. 6C). Full PD-1 blockade was achieved at doses ≥1 mg/kg every 3 weeks (Q3W). DART-D administrations was associated with enhanced proliferation of peripheral CD8⁺ T cells, but no associated changes in T_{reg} population. A dose-dependent upregulation of ICOS was observed on circulating CD4⁺ T cells (FIG. 6D) was observed. ICOS upregulation, a surrogate measure of CTLA-4 blockade, was induced by DART-D at doses ≥3 mg/kg. An association between the ICOS biomarker and objective clinical responses in the study (FIG. 6E) suggests that CTLA-4 blockade, rather than depletion of CTLA-4⁺ cells, drives the clinical benefits of combination therapy.

In the ongoing Dose Escalation Phase, DART-D was generally well-tolerated at doses up to the top predefined dose level of 10 mg/kg. The safety of all dose levels was evaluated in a 3+3+3 dose escalation study design. At doses ≥3 mg/kg, DART-D demonstrated evidence of anti-tumor activity surpassing expectations of anti-PD-1 monotherapy. Additional patients were allocated to select escalation cohorts to generate further clinical data at dose levels of interest. Among 33 patients treated, treatment-related adverse events (TRAEs) occurred in 26/33 (78.8%) patients, most commonly fatigue (24%), nausea, arthralgia, pruritus, and rash (18% each). The rate of Grade ≥3 TRAEs was 24.2%. Treatment-related serious adverse events included enteritis, enterocolitis, pneumonitis, and myocarditis (n=1 each) and occurred at dose levels between 3 and 10 mg/kg; all patients recovered without sequelae after appropriate treatment. Infusion-related reactions (IRRs) were observed, all mild to moderate in severity.

Among 25 response-evaluable patients, objective responses were observed in 4 patients with tumor types conventionally unresponsive to checkpoint inhibition. Responders include patients with microsatellite-stable colorectal cancer, metastatic type AB thymoma (both confirmed partial responses (PRs)), anti-PD-L1-refractory serous fallopian tube carcinoma (unconfirmed PR with >50% reduction of CA-125), and metastatic castration-resistant prostate cancer (confirmed complete response (CR)) with resolution of elevated pre-treatment prostate-specific antigen). Nine patients had stable disease as a best response. All responding patients (n=4) were among 13 response-evaluable patients treated at doses ≥3 mg/kg (FIG. 7), and demonstrated ICOS upregulation on circulating CD4⁺ T cells (FIG. 6E). These data support additional dose levels between about 3.0 mg/kg and about 10.0 mg/kg, particularly between about 6.0 mg/kg and 10 mg/kg. The encouraging clinical data suggest that safe and effective dual checkpoint blockade with DART-D may offer improved clinical benefit to advanced cancer patients. These initial observations indicate that the purpose-designed, multi-specific biomolecule tested displays clinical activity, convenient administration, and demonstrates less toxicity than a combination of individual therapeutic mAbs.

During the Dose Escalation Phase it was determined the maximum administered dose (MAD) of DART-D was 10 mg/kg. The maximum tolerated dose (MTD) was not exceeded or defined. Based on the totality of the clinical, PK, and pharmacodynamic data a recommended Phase 2 dose of 6 mg/kg was selected for evaluation in the Cohort Expansion Phase. Additionally, based on the DART-D safety profile and to ensure a more consistent study, drug exposure throughout the treatment course administration was changed such that DART-D is administered Q3W throughout the treatment period (108 weeks, or until disease progress or toxicity that necessitates discontinuation). This study is ongoing and data is still maturing.

### Example 4

### Materials and Methods

Materials and methods are provided below and in the Figure Descriptions above.

Ligand blockade: Jurkat/PD-1, Jurkat/CTLA-4 and Jurkat/PD-1+CTLA-4 were generated by stable transfection of parental cells. Cells were incubated with 1 ug/mL biotinylated recombinant B7-1 or PD-L1 (BPS Bioscience, San Diego, USA) in the presence of unlabeled test molecules and detected with Streptavidin/R-PE. Flow cytometry was performed using FACSCanto II cytometer (BD Biosciences, San Jose, USA) in plate format; at least 20,000 events were collected for test well.

Dimerization assay: The PathHunter® dimerization assay (DiscoveRx, Fremont, USA) utilizes the enzyme fragment complementation technology where two split β gal fragments, which independently had no enzymatic activity, could be formed back into a functional β gal to generate chemiluminescence. U2OS cells were engineered to stably coexpress the fragment-tagged CTLA-4 and PD-1 and the dimerization assay was performed in the presence of test articles.

Engineered Reporter Assay: PD-1, CTLA-4 and PD-1+CTLA-4 bioassay systems were obtained from Promega (Madison, USA) and used according to manufacturers' instructions. A CHO-based stimulator line expressing anti-CD3 and checkpoint ligands (PD-L1, B7-1 or both) and a Jurkat-based reporter cell line were cultured together in the presence of DART-D or mAbs. Induction of luciferase under control of NF-AT or IL-2 promoter was detected using Bio Glo substrate.

Primary SEB Assay: Cryopreserved healthy donor PBMC were thawed and plated 10⁵ cells/well in 200 μL of complete RPMI. mAbs and bispecific inhibitors were added at fixed concentration (10 ug/mL), and Staph Aureus Enterotoxin B (SEB, Toxin Technology, Inc., Sarasota, USA) was titrated as indicated. Cells were incubated for 96 hours prior to supernatant collection and evaluation of secreted IL-2.

Antibody-dependent depletion of Tregs: Freshly isolated PBMCs were plated in complete RPMI at 10⁶ cells/mL and stimulated with CD3 beads (Invitrogen, Carlsbad, USA) in the presence of indicated test articles (e.g., mAbs or DART-D at 1 μg/mL). 48 hours later cells were collected and stained with CD4 and FoxP3 mAbs.

Cynomolgus Monkey Toxicity Study: The non-clinical toxicology study was conducted in accordance with the US Department of Agriculture Animal Welfare Act (9 CFR Parts 1, 2, and 3), and the Guide for the Care and Use of Laboratory Animals, Institute of Laboratory Animal Resources. A 4-week, repeat-dose study was conducted in cynomolgus monkeys (Macaca fascicularis) to evaluate the toxicity of DART-D. After the completion of dosing, a subset of animals (2/sex/group) underwent a 10-week recovery period to evaluate the persistence or delayed occurrence of effects. Forty cynomolgus monkeys of Chinese origin were randomly assigned to 4 groups (5/sex/group) to achieve similar group mean body weights. The animals were dosed with the vehicle (5% dextrose injection) or DART-D via intravenous (IV) infusion for 30 minutes once weekly for a total of 4 doses (days 1, 8, 15, and 22). The DART-D dose levels were 10, 40, or 100 mg/kg/dose. Evaluation of animals, including electrocardiographic, vital signs assessments, hematology, urinalysis PK, ADA and immunophenotyping were performed periodically. A full necropsy was conducted for all animals, with organs weighed and tissues collected, preserved, and processed for histopathologic evaluation. Samples of spleen were collected from each animal for splenocyte immunophenotyping.

DART-D PK studies: Intact DART-D serum concentrations were measured by bispecific enzyme-linked immunosorbent assay at indicated timepoints. Open one- or two-compartment IV infusion model was employed to fit the PK data using actual times and concentrations, actual infusion times, and nominal doses. Individual first dose data were modeled and weighted reciprocally of predicted concentration squared (−2). For receptor occupancy studies, the following Eₘₐₓ model was used: E=(Eₘₐₓ*C)/(EC₅₀+C); where E=% RO, Eₘₐₓ=maximal % RO, EC50=concentration producing half maximal effect, and C=concentration of DART-D. For PK simulations mean values of best estimates of the model parameters were used for a potential clinical dose range of 3 mg/kg to 10 mg/kg and Q3W infusions.

Receptor Occupancy (RO): One hundred microliter (L) volumes of whole blood samples (per time point/per patient) were incubated with saturating concentration of DART-D, followed by lysis and detection of DART-D by biotinylated anti-drug mAb/Strep-PE in “DART-D-spiked” and control samples. After subtraction of background fluorescence (Step-PE only), RO values were calculated as a fraction of maximal binding capacity: RO=(untreated MFI(PE)−background MFI(PE))/(treated MFI(PE)−background MFI(PE)).

Table 3 presents a list of flow cytometry reagents used in the studies described herein.

TABLE 3
Cytometry Reagents

| Antigen/Fluorophore | Clone | Supplier |
|---|---|---|
| FoxP3/FITC | PCH101 | Invitrogen |
| CD3/V500 | SP34-2 | BD Biosciences |
| CD4/APC-H7 | SK3 | BD Biosciences |
| CD8/FITC | RPA-T8 | BD Biosciences |
| CD45/PerCP-Cy5.5 | HI30 | BD Biosciences |
| PD-1/APC | J105 | eBiosciences |
| CTLA-4/Dazzle-594 | BNI3 | Biolegend |
| ICOS/PE-Cy7 | C398.4A | Biolegend |
| Ki67/AlexaFluor488 | B56 | eBiosciences |
| CD25/BB515 | 2A3 | BD Biosciences |
| cCD45/APC | 30-F11 | Invitrogen |
| CD28/PE | CD28.2 | BD Biosciences |
| CD95/V450 | DX2 | BD Biosciences |
| Streptavidin/R-PE | — | Life Science |

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A method of treating a cancer comprising administering a PD-1×CTLA-4 bispecific molecule to a subject in need thereof, wherein said PD-1×CTLA-4 bispecific molecule comprises a PD-1 Binding Domain and a CTLA-4 Binding Domain, and wherein said method comprises administering said PD-1×CTLA-4 bispecific molecule to a subject at a dose of about 3 mg/kg to about 10 mg/kg once every 3 weeks.

2. A method of stimulating immune cells comprising administering a PD-1×CTLA-4 bispecific molecule to a subject in need thereof, wherein said PD-1×CTLA-4 bispecific molecule comprises a PD-1 Binding Domain and a CTLA-4 Binding Domain, and wherein said method comprises administering said PD-1×CTLA-4 bispecific molecule to a subject at a dose of about 3 mg/kg to about 10 mg/kg once every 3 weeks.

3. The method of claim 1 or 2, wherein said PD-1×CTLA-4 bispecific molecule is administered to said subject at a dose of about 3 mg/kg to about 10 mg/kg once every 3 weeks during an induction period.

4. The method of claim 2 or 3, wherein said immune cells are T cells.

5. The method of any one of claims 1-4, wherein:
(I) said PD-1 Binding Domain comprises a Light Chain Variable Domain (VL_{PD-1}) that comprises the CDR_{L}1, CDR_{L}2 and CDR_{L}3 of SEQ ID NO:1, and a Heavy Chain Variable Domain (VH_{PD-1}) that comprises the PD-1-specific CDR_{H}1, CDR_{H}2 and CDR_{H}3 of SEQ ID NO:5; and
(II) said CTLA-4 Binding Domain comprises a Light Chain Variable Domain (VL_{CTLA-4}) that comprises the CDR_{L}1, CDR_{L}2 and CDR_{L}3 of SEQ ID NO:9, and a Heavy Chain Variable Domain (VH_{CTLA-4}) that comprises the CTLA-4-specific CDR_{H}1, CDR_{H}2 and CDR_{H}3 of SEQ ID NO:13.

6. The method of any one of claims 1-5, wherein said PD-1×CTLA-4 bispecific molecule comprises:
(I) two of said PD-1 Binding Domains; and
(II) two of said CTLA-4 Binding Domains.

7. The method of any one of claims 1-6, wherein:
(a) said PD-1 Binding Domain comprises the VL Domain of SEQ ID NO:1 and the VH Domain of SEQ ID NO:5; and
(b) said CTLA-4 Binding Domain comprises the VL Domain of SEQ ID NO:9 and the VH Domain of SEQ ID NO:13.

8. The method of any one of claims 1-7, wherein said PD-1×CTLA-4 bispecific molecule comprises a Hinge Domain and an Fc Region of an IgG1, IgG2, IgG3, or IgG4 isotype.

9. The method of claim 8, wherein said Fc Region and said Hinge Doman are of the IgG4 isotype, and wherein said Hinge Domain comprises a stabilizing mutation.

10. The method of any one of claims 8-9, wherein said Fc Region is a variant Fc Region that comprises:
(a) one or more amino acid modifications that reduces the affinity of the variant Fc Region for an FcγR; and/or
(b) one or more amino acid modifications that enhances the serum half-life of the variant Fc Region.

11. The method of claim 10, wherein:
(a) said one or more amino acid modifications that reduces the affinity of the variant Fc Region for an FcγR comprise the substitution of L234A or L235A, or L234A and L235A; and/or
(b) said one or more amino acid modifications that enhances the serum half-life of the variant Fc Region comprise the substitution of M252Y;
or M252Y and S254T; or M252Y and T256E; or M252Y, S254T and T256E; or K288D and H435K,
wherein said numbering is that of the EU index as in Kabat.

12. The method of any one of claims 1-11, wherein said PD-1×CTLA-4 bispecific molecule is a diabody comprising one polypeptide chain that comprises the amino acid sequence of SEQ ID NO:40 and a second polypeptide chain that comprises the amino acid sequence of SEQ ID NO:41.

13. The method of any one of claims 1-12, wherein said PD-1×CTLA-4 bispecific molecule is a diabody comprising two polypeptide chains each comprising the amino acid sequence of SEQ ID NO:40 and two polypeptide chains each comprising the amino acid sequence of SEQ ID NO:41.

14. The method of any one of claims 1-16, wherein said PD-1×CTLA-4 bispecific molecule is administered at a dose of between about 3 mg/kg and 8 mg/kg.

15. The method of any one of claims 1-14, wherein said PD-1×CTLA-4 bispecific molecule is administered at a dose of about 6 mg/kg.

16. The method of any one of claims 3-15, further comprising administering said PD-1×CTLA-4 bispecific molecule to said subject at a dose of from about 3 mg/kg to about 10 mg/kg once every 6 weeks during a maintenance period, wherein said maintenance period follows said induction period.

17. The method of any one of claims 3-13 or 16, wherein said induction period has a duration of up to about 24 weeks.

18. The method of any one of claims 3-13, or 16-17, wherein said maintenance period has a duration of up to about 84 weeks.

19. The method of any one of claims 3-13, or 16-18, wherein said PD-1×CTLA-4 bispecific molecule is administered at a dose of between about 3 mg/kg and 8 mg/kg during said induction period.

20. The method of any one of claims 3-13, or 16-19, wherein said PD-1×CTLA-4 bispecific molecule is administered at a dose of about 6 mg/kg during said induction period.

21. The method of any one of claims 16-20, wherein said PD-1×CTLA-4 bispecific molecule is administered at a dose of between about 3 mg/kg and 8 mg/kg during said maintenance period.

22. The method of any one of claims 16-21, wherein said PD-1×CTLA-4 bispecific molecule is administered at a dose of about 6 mg/kg during said maintenance period.

23. The method of any one of claims 16-22, wherein said dose of said PD-1×CTLA-4 bispecific molecule administered in said maintenance period is the same as said dose administered in said induction period.

24. The method of any one of claims 1-23, wherein said PD-1×CTLA-4 bispecific molecule is administered by intravenous (IV) infusion.

25. The method of any one of claims 1-24, wherein said cancer is selected from the group consisting of: an adrenal gland cancer, an AIDS-associated cancer, an alveolar soft part sarcoma, an astrocytic tumor, an anal cancer, a bile duct cancer, a bladder cancer, a bone cancer, a brain cancer, a brain and spinal cord cancer, a breast cancer, a HER2+ breast cancer, a triple negative breast cancer (TNBC), a carotid body tumors, a cervical cancer, an HPV-related cervical cancer, a cervical squamous cell carcinoma, a chondrosarcoma, a chordoma, a clear cell carcinoma, a colon cancer, a colorectal cancer (CRC), a microsatellite instability-high colorectal cancer (MSI-H CRC), a microsatellite-stable colorectal cancer (non-microsatellite-instability-high colorectal cancer, non-MSI-H CRC), a desmoplastic small round cell tumor, an endometrial cancer, an ependymoma, a Ewing's tumor, an extraskeletal myxoid chondrosarcoma, a fallopian tube carcinoma, a fibrogenesis imperfecta ossium, a fibrous dysplasia of the bone, a gallbladder or bile duct cancer, a gastric cancer, a gestational trophoblastic disease, a germ cell tumor, a glioblastoma, a head and neck cancer, an HPV-related head and neck cancer, a hematological malignancy, a hepatocellular carcinoma, an islet cell tumor, a Kaposi's Sarcoma, a kidney cancer, a leukemia, a liposarcoma/malignant lipomatous tumor, a liver cancer, a lymphoma, a lung cancer, a non-small-cell lung cancer (NSCLC), a medulloblastoma, a melanoma, a meningioma, Merkel cell carcinoma, a mesothelioma pharyngeal cancer, a multiple endocrine neoplasia, a multiple myeloma, a myelodysplastic syndrome, a neuroblastoma, a neuroendocrine tumor, an ovarian cancer, a pancreatic cancer, a papillary thyroid carcinoma, a parathyroid tumor, a pediatric cancer, a peripheral nerve sheath tumor, a pheochromocytoma, a pituitary tumor, a prostate cancer, a metastatic castration resistant prostate cancer (mCRPC), a posterior uveal melanoma, a renal cancer, a renal cell carcinoma (RCC), a rhabdoid tumor, a rhabdomyosarcoma, a sarcoma, a skin cancer, a small round blue cell tumor of childhood (including neuroblastoma and rhabdomyosarcoma), a soft-tissue sarcoma, a pleomorphic undifferentiated sarcoma, a dedifferentiated liposarcoma, a synovial sarcoma, a myxofibrosarcoma, a squamous cell cancer, a squamous cell cancer of the head and neck (SCCHN), a stomach cancer, a synovial sarcoma, a testicular cancer, a thymic carcinoma, a thymoma, a thyroid cancer, a thyroid metastatic cancer, and a uterine cancer.

26. The method of claim 25, wherein said cancer is selected from the group consisting of:
cervical cancer, HPV-related cervical cancer, cervical squamous cell carcinoma, CRC, MSI-H CRC, non-MSI-H CRC, head and neck cancer, HPV-related head and neck cancer, lung cancer, melanoma, NSCLC, prostate cancer, renal cancer, RCC, soft-tissue sarcoma, a pleomorphic undifferentiated sarcoma, a dedifferentiated liposarcoma, a synovial sarcoma, a myxofibrosarcoma, squamous cell cancer, and SCCHN.

27. The method of any one of claims 1-26, further comprising administering a therapeutically or prophylactically effective amount of one or more additional therapeutic agents or chemotherapeutic agents.

28. The method of any one of claims 1-27, wherein said subject in need thereof is a human.

29. A pharmaceutical kit comprising:
(a) a container comprising a PD-1×CTLA-4 bispecific molecule; and
(b) an instructional material,
wherein the instructional material instructs that said PD-1×CTLA-4 bispecific molecule is to be used according to the method of any one of claims 1-27.

30. Use of the pharmaceutical kit of claim 29 for the treatment of cancer or for stimulating immune cells.


SEQUENCE LISTING (42 sequences)

SEQ ID NO: 1 - protein, 111 aa, Artificial Sequence
  Description of Artificial Sequence Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIK

SEQ ID NO: 2 - protein, 15 aa, Artificial Sequence
  Description of Artificial Sequence Synthetic peptide
RASESVDNYGMSFMN

SEQ ID NO: 3 - protein, 7 aa, Artificial Sequence
  Description of Artificial Sequence Synthetic peptide
AASNQGS

SEQ ID NO: 4 - protein, 9 aa, Artificial Sequence
  Description of Artificial Sequence Synthetic peptide
QQSKEVPYT

SEQ ID NO: 5 - protein, 119 aa, Artificial Sequence
  Description of Artificial Sequence Synthetic polypeptide
QVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSS

SEQ ID NO: 6 - protein, 5 aa, Artificial Sequence
  Description of Artificial Sequence Synthetic peptide
SYWMN

SEQ ID NO: 7 - protein, 17 aa, Artificial Sequence
  Description of Artificial Sequence Synthetic peptide
VIHPSDSETWLDQKFKD

SEQ ID NO: 8 - protein, 10 aa, Artificial Sequence
  Description of Artificial Sequence Synthetic peptide
EHYGTSPFAY

SEQ ID NO: 9 - protein, 108 aa, Artificial Sequence
  Description of Artificial Sequence Synthetic polypeptide
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIK

SEQ ID NO: 10 - protein, 12 aa, Artificial Sequence
  Description of Artificial Sequence Synthetic peptide
RASQSVSSSFLA

SEQ ID NO: 11 - protein, 7 aa, Artificial Sequence
  Description of Artificial Sequence Synthetic peptide
GASSRAT

SEQ ID NO: 12 - protein, 9 aa, Artificial Sequence
  Description of Artificial Sequence Synthetic peptide
QQYGSSPWT

SEQ ID NO: 13 - protein, 118 aa, Artificial Sequence
  Description of Artificial Sequence Synthetic polypeptide
QVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSS

SEQ ID NO: 14 - protein, 5 aa, Artificial Sequence
  Description of Artificial Sequence Synthetic peptide
SYTMH

SEQ ID NO: 15 - protein, 17 aa, Artificial Sequence
  Description of Artificial Sequence Synthetic peptide
FISYDGSNKHYADSVKG

SEQ ID NO: 16 - protein, 9 aa, Artificial Sequence
  Description of Artificial Sequence Synthetic peptide
TGWLGPFDY

SEQ ID NO: 17 - protein, 8 aa, Artificial Sequence
  Description of Artificial Sequence Synthetic peptide
GGGSGGGG

SEQ ID NO: 18 - protein, 6 aa, Artificial Sequence
  Description of Artificial Sequence Synthetic peptide
GGCGGG

SEQ ID NO: 19 - protein, 28 aa, Artificial Sequence
  Description of Artificial Sequence Synthetic peptide
EVAALEKEVAALEKEVAALEKEVAALEK

SEQ ID NO: 20 - protein, 28 aa, Artificial Sequence
  Description of Artificial Sequence Synthetic peptide
KVAALKEKVAALKEKVAALKEKVAALKE

SEQ ID NO: 21 - protein, 28 aa, Artificial Sequence
  Description of Artificial Sequence Synthetic peptide
EVAACEKEVAALEKEVAALEKEVAALEK

SEQ ID NO: 22 - protein, 28 aa, Artificial Sequence
  Description of Artificial Sequence Synthetic peptide
KVAACKEKVAALKEKVAALKEKVAALKE

SEQ ID NO: 23 - protein, 5 aa, Artificial Sequence
  Description of Artificial Sequence Synthetic peptide
ASTKG

SEQ ID NO: 24 - protein, 217 aa, Homo sapiens
  MOD_RES (217)..(217): K or absent
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 25 - protein, 216 aa, Homo sapiens
  MOD_RES (216)..(216): K or absent
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGX

SEQ ID NO: 26 - protein, 217 aa, Homo sapiens
  MOD_RES (217)..(217): K or absent
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGX

SEQ ID NO: 27 - protein, 217 aa, Homo sapiens
  MOD_RES (217)..(217): K or absent
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 28 - protein, 217 aa, Artificial Sequence
  Description of Artificial Sequence Synthetic polypeptide
  MOD_RES (217)..(217): K or absent
APEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGX

SEQ ID NO: 29 - protein, 217 aa, Artificial Sequence
  Description of Artificial Sequence Synthetic polypeptide
  MOD_RES (217)..(217): K or absent
APEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGX

SEQ ID NO: 30 - protein, 4 aa, Artificial Sequence
  Description of Artificial Sequence Synthetic peptide
GGGS

SEQ ID NO: 31 - protein, 6 aa, Artificial Sequence
  Description of Artificial Sequence Synthetic peptide
LGGGSG

SEQ ID NO: 32 - protein, 6 aa, Artificial Sequence
  Description of Artificial Sequence Synthetic peptide
LEPKSS

SEQ ID NO: 33 - protein, 5 aa, Artificial Sequence
  Description of Artificial Sequence Synthetic peptide
APSSS

SEQ ID NO: 34 - protein, 8 aa, Artificial Sequence
  Description of Artificial Sequence Synthetic peptide
APSSSPME

SEQ ID NO: 35 - protein, 10 aa, Homo sapiens
DKTHTCPPCP

SEQ ID NO: 36 - protein, 15 aa, Homo sapiens
EPKSCDKTHTCPPCP

SEQ ID NO: 37 - protein, 12 aa, Homo sapiens
ERKCCVECPPCP

SEQ ID NO: 38 - protein, 12 aa, Homo sapiens
ESKYGPPCPSCP

SEQ ID NO: 39 - protein, 12 aa, Artificial Sequence
  Description of Artificial Sequence Synthetic peptide
ESKYGPPCPPCP

SEQ ID NO: 40 - protein, 499 aa, Artificial Sequence
  Description of Artificial Sequence Synthetic polypeptide
EIVLTQSPATLSLSPGERATLSCRASESVDNYGMSFMNWFQQKPGQPPKLLIHAASNQGSGVPSRFSGSGSGTDFTLTISSLEPEDFAVYFCQQSKEVPYTFGGGTKVEIKGGGSGGGGQVQLVESGGGVVQPGRSLRLSCAASGFTFSSYTMHWVRQAPGKGLEWVTFISYDGSNKHYADSVKGRFTVSRDNSKNTLYLQMNSLRAEDTAIYYCARTGWLGPFDYWGQGTLVTVSSGGCGGGEVAACEKEVAALEKEVAALEKEVAALEKESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 41 - protein, 269 aa, Artificial Sequence
  Description of Artificial Sequence Synthetic polypeptide
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPWTFGQGTKVEIKGGGSGGGGQVQLVQSGAEVKKPGASVKVSCKASGYSFTSYWMNWVRQAPGQGLEWIGVIHPSDSETWLDQKFKDRVTITVDKSTSTAYMELSSLRSEDTAVYYCAREHYGTSPFAYWGQGTLVTVSSGGCGGGKVAACKEKVAALKEKVAALKEKVAALKE

SEQ ID NO: 42 - protein, 13 aa, Artificial Sequence
  Description of Artificial Sequence Synthetic peptide
GGGDKTHTCPPCP